(19) United States
(12) Patent Application Publication
(10) Pub. No.: US 2022/0228126 A1
(43) Pub. Date: Jul. 21, 2022

(54) USE OF PCBP1 TO GENERATE INDUCED PLURIPOTENT STEM CELLS WHILE INHIBITING ONCOGENESIS

(71) Applicant: Ibex Biosciences, LLC, Cumberland, MD (US)

(72) Inventors: Norman Zhennan LAI, N. Potomac, MD (US); Alberto Murat CROCI, Washington, DC (US); Michael Joseph KARLIN, Bethesda, MD (US); Vidal Felix DE LA CRUZ, JR., Phoenixville, PA (US); Yuebin TAN, Cumberland, MD (US)

(21) Appl. No.: 17/591,309

(22) Filed: Feb. 2, 2022

Related U.S. Application Data
(63) Continuation of application No. PCT/US2020/045477, filed on Aug. 7, 2020.
(60) Provisional application No. 62/883,815, filed on Aug. 7, 2019.

(51) Int. Cl. C12N 5/074; A61K 38/17

(52) U.S. Cl. CPC C12N 5/0696; A61K 38/1709; C12N 2506/08; C12N 2506/1307; C12N 2501/604; C12N 2501/603; C12N 2501/60; C12N 2510/00; C12N 2501/602

(57) ABSTRACT

The present disclosure provides compositions and methods of using gene therapy to create induced pluripotent stem cells (iPSCs) without inducing cancer or tumorigenesis. The methods disclosed herein employ plasmids and vectors that contain transcription factors and an anti-oncogene such as PCBP1 which inhibits the expression of cancer biomarkers and concomitant oncogenesis.

### CROSS-REFERENCE TO RELATED APPLICATIONS

This application is a continuation of International Application No. PCT/US2020/045477, filed Aug. 7, 2020, which claims the benefit of U.S. Provisional Application No. 62/883,815 filed Aug. 7, 2019, the contents of which are hereby incorporated by reference in their entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing, filename: IBEX-002_06US SeqList_ST25.txt, date created: Feb. 1, 2022, file size: 7, 455 bytes.

### FIELD

The present disclosure relates to compositions and methods to induce pluripotent stem cells while inhibiting oncogenesis. Specifically, the transduction of differentiated cells with one or more stem cell transcription factors and/or PCBP1 results in the formation of iPSCs from differentiated cells, but the presence of an anti-oncogene such as PCBP1 inhibits the formation of tumors and the development of cancer that can be caused by use of such stem cell transcription factors. The compositions may be administered in vitro, in vivo, or in situ.

### BACKGROUND

Stem cells are defined by their capacity for self-renewal and ability to differentiate into a variety of somatic cell types. Cellular programs regarding proliferation, potency, and cell fate determination can be mediated by signal transduction events that modulate transcription factor expression and/or activation.

Stem cells have tremendous promise to understand and treat a range of diseases, injuries and other health-related conditions. Perhaps the most important potential application of human stem cells is the generation of cells and tissues that could be used for cell-based therapies. Today, donated organs and tissues are often used to replace ailing or destroyed tissue, but the need for transplantable tissues and organs far outweighs the available supply. Stem cells, directed to differentiate into specific cell types, offer the possibility of a renewable source of replacement cells and tissues to treat diseases including, but not limited to, macular degeneration, spinal cord injury, stroke, burns, heart disease, diabetes, osteoarthritis, and rheumatoid arthritis.

To realize the promise of novel cell-based therapies, the stem cells must be manipulated so that they possess the necessary characteristics for successful differentiation, transplantation, and engraftment. For example, to be useful for transplant purposes, stem cells must be reproducibly made to proliferate extensively and generate sufficient quantities of cells for making tissue, differentiate into the desired cell type(s), survive in the recipient after transplant, integrate into the surrounding tissue after transplant, function appropriately for the duration of the recipient's life, and avoid immune rejection.

### SUMMARY OF THE INVENTION

One of the major hurdles in successfully using stem cell therapies is that these cells, once implanted, may become cancerous. Further, stem cell therapies known in the art require ex vivo modifications prior to infusion or implantation in order to make a successful therapeutic product. While this is typically done in the laboratory on isolated cells, the present disclosure provides an approach for generating induced pluripotent stem cells (iPSCs) in situ, e.g. directly in the organ.

Here, gene therapy is used to introduce a PCBP1, a variant, or a mutant thereof, alone or with one or more transcription factors to the tissue in the body to induce the formation of stem cells.

The present disclosure provides methods of inducing pluripotent stem cells from differentiated cells comprising introducing a vector comprising the nucleic acid sequence of a PCBP1, a variant, or a mutant thereof, wherein induction of the pluripotent stem cells is not accompanied by tumorigenesis. The present disclosure also provides methods of inducing pluripotent stem cells from differentiated cells comprising introducing a vector comprising the nucleic acid sequences of a PCBP1, a variant, or a mutant thereof and one or more other transcription factors, wherein induction of the pluripotent stem cells is not accompanied by tumorigenesis.

In some embodiments, the methods are performed in vitro. In some embodiments, the methods are performed in vivo. In some embodiments, the methods are performed ex vivo. In some embodiments, the methods are performed in situ.

In some embodiments the differentiated cells are mammalian. In some embodiments the mammalian cells are human. In some embodiments the mammalian cells are organ cells, tissue cells, or blood cells. In some embodiments the tissue cells are retinal cells.

In some embodiments, the vector comprises the nucleic acid sequence of PCBP1, or a variant or mutant thereof. In some embodiments the vector comprises the nucleic acid sequences of PCBP1, or a variant or mutant thereof, and at least one of the transcription factors selected from the group consisting of SOX2, OCT4, and KTL4. In some embodiments the vector comprises the nucleic acid sequences of PCBP1, or a variant or mutant thereof and at least two of the transcription factors selected from the group consisting of SOX2, OCT4, and KTL4. In some embodiments the vector comprises the nucleic acid sequences of PCBP1 or a mutant or variant thereof, SOX2, OCT4, and KTL4.

### BRIEF DESCRIPTION OF THE FIGURES

FIG. 1A-C show examples of vectors useful in the present disclosure. The therapeutic gene vector may encode PCBP1 and stem cell inducing transcription factors (Sox2, Oct4, and Klf4). This vector system uses the human elongation factor-1 alpha (EFP promoter to drive expression of genes fused with a green fluorescent protein (GFP) via a cleavable protein segment. This LVV vector system was then transduced into CFs. As demonstrated in Example 1, PCBP1 down-regulates and de-activates PRL3, a downstream protein involved in tumorigenesis, and thus provides a useful safe-guard for clinical applications in reducing oncogenic side effects. TRE: Tetracycline (SEQ ID NO: 5); rtTA: reverse tetracycline-controlled trans activator; tTS: transcription silencer is a fusion of the Tet Repressor Protein (TetR) with a KRAB silencing domain. tTS can be used to significantly lower the basal expression from 1^{st} generation Tet-On Inducible Expression Systems. In the absence of doxycycline, tTS binds to the tet-operator sequences within TRE promoters, silencing gene expression. Upon addition of doxycycline, tTS undergoes a conformational change that causes its release from the promoter, to be replaced by the Tet-On transactivator.

FIG. 2A shows a comparison of GFP cell counts in retinal cell (ARPE-19, ATCC™) cultures after transfection with therapeutic genes in vitro. All GFP positive cell numbers are estimated based on 10³ cells.

FIG. 2B shows retinal cells transfected by the therapeutic gene vectors coexpressing green fluorescent protein and monitored by a fluorescence microscopy (10×). The results (photos) indicate that PCBP1-GFP, Oct4, Sox2, and Klf4 were successfully delivered by the therapeutic vector system and co-expressed in the transfected cells.

FIG. 3 shows real-time PCR results to compare the mRNA level of PCBP1 between cells transfected by the gene therapy (GT) vector and control cells without GT. PCBP1 mRNA levels in GT transfected cells are more than 3 fold higher (ddCt=3.37) than that of the control cells.

FIG. 4A-B shows immunohistochemistry staining of a biomarker of retinal stem cells by antibody to human CRX-1 (see red fluorescent light). This indicates that some of the cells have been turned into retinal stem cell-like cells after differentiated retinal cells were transduced with a vector including PCBP1 and the SOX2, OCT4, KLF4 transcription factors.

FIG. 5A-D shows immunohistochemistry staining of a biomarker of retinal stem cells by the antibody to human SOX2 (red fluorescent light). Multiple staining was performed. First, the retinal stem cell biomarker of CRX was detected. Then, the cells transduced by the PCBP1-vector and those transduced by vector SOX 2 were stained to show that these cells overlap with CRX expression. This indicates that some of the cells have been turned into retinal stem cell-like cells after differentiated cells were transduced with a vector containing PCBP1 and the SOX2, OCT4, KLF4 transcription factors.

FIG. 6A-B shows skin cancer cells transfected with either the control (GFP only) or PCBP1-GFP (FIG. 6A). Western-blot analysis on skin cancer cells treated with PCBP1 and its mutants that can down-regulate the protein level of the PRL-3, an oncogenic factor in cancer cells. Lane 1: GFP only; lane 2: PCBP1 with one mutation as mPCBP1_1; Lane 3: PCBP1 wide-type as wPCBP1_0; Lane 4: PCBP1 with two mutations as mPCBP1_2. The beta actin was served as a control set of house-keeping protein in this assay.

FIG. 7 shows real-time qRT-PCR reactions to detect the expression level of both PCBP1 and PRL-3 in the cancer cells. The left panel indicates that PCBP1 was highly expressed in the Treatment group. The right panel shows that the PRL-3 was significantly down-regulated in the Treatment group of the cancer cells. The untreated groups were transduced by a GFP vector only.

FIG. 8 shows representative schematics of experimental designs.

FIG. 9 shows a representative delivery method to deliver drugs into the retinal layer of the eye (red square). See Park et al. (2015)

FIG. 10 shows an example of the in vivo pre-clinical study to test visual acuity after gene therapy on macular degenerative disease. See Lu et al. (2009).

FIG. 11A-B shows a representative experimental design to demonstrate PCBP1 inhibits expression of biomarkers associated with metastasis and cancer (e.g. PRL-3/STAT3).

FIG. 12 shows a putative mechanism by which PCBP1 inhibits PRL-3/STAT3 expression.

FIG. 13A-B shows results from transfection of cardiac fibroblasts (CFs) with the vectors of the present disclosure. FIG. 13A shows CFs transduced with LVVs expressing PCBP1 and GFP. GFP expression is detected by fluorescent microscopy (×10). (a): PCBP1-IRES-GFP (b): PCBP1-IRES-GFP and Sox2/Oct4/Klf4 (P+SOK) co-transduction (c):LV vector without GFP as control; (d) GFP vector control. GFP was detected under the fluorescent microscope to count the transduction efficiency with about more than 80% cells transduced. FIG. 13B shows a comparison of the PCBP1 mRNA levels between the CFs transduced by the PCBP1-IRES-GFP vector and control (no transfection) by qRT-PCR. PCBP1 expression is more than 99-fold higher in the treatment group (ddCt=99.9); p<0.05.

FIG. 14A-F shows immunohistochemistry staining for a cardiac stem cell biomarker. The CFs were transduced and labeled using antibody against human c-Kit and detected with NorthernLights-conjugated secondary antibody (A, B). No transduction control group under red fluorescent filter (A) and bright field (B); (C, D) PCBP1-IRES-GFP (c-Kit staining) under red fluorescent filter (C) and bright field (D); PCBP1-IRES-GFP and transcription factors Sox2, Oct4, Klf4 (P+SOK) co-transduction group (c-Kit staining) observed under red fluorescent filter (E) and bright field (F). Insets showing areas of interest enlarged for details. Scale Bar: 100 μm.

FIG. 15 shows real time RT-PCR reactions to detect mRNA expression of cardiac biomarkers after iPSCs induction. Top to bottom: COL1a1, c-Kit, TNNT2. Left column: LVV PCBP1-GFP and Sox2/Oct4/Klf4 (P+SOK) co-transduction; Right column: no transduction control. *p<0.05.

FIG. 16 shows an exemplary animal model for testing the systems of the present disclosure in a murine model of myocardial infarction.

FIG. 17A-B: Flow cytometry detection of CD44 and CD24 in MCF-7 and MCF-7 (CSC) (BCSC). FIG. 17A: CD44 staining. FIG. 17B: CD24 staining.

FIG. 18A-D: FACS analysis on biomarkers confirmed by dual-color scatter plot (CD44 vs CD24). Flow cytometry detection of CD24 and CD44 in MCF-7 and MCF-7 (CSC) via dual-color scatter plot. FIG. 18A: isotype control of MCF-7 (CSC), FIG. 18B: CD44-PE & CD24-APC staining of MCF-7 (CSC), FIG. 18C: isotype control of MCF-7, FIG. 18D: CD44-PE & CD24-APC staining of MCF-7. These data are same from FIG. 17 analyzed by dual-color scatter plot for additional clarity.

FIG. 19A-D: Flow cytometry detection of CD133 in MCF-7 (CSC) and MDA-MB-231. FIG. 19A: isotype control, FIG. 19B: MCF-7 (CSC), FIG. 19C: MDA-MB-231, FIG. 19D: merge. Cells were harvested stained with Anti-Human CD133 APC-conjugated Monoclonal Antibody (10 uL/10⁶ cells). Non-specific antibody used as Isotype control.

FIG. 20: Western blot (WB) analysis of CD133, CD44 and CD24 in MCF-7 and U87-MG treated with or without TGF-beta. Lane 1: MCF-7 (without TGF-beta), lane 2: MCF-7 (with TGF-beta); lane 3: U87-MG (without TGF-beta), lane 4: U87-MG (with TGF-beta).

FIG. 21A-C: Normalized by GAPDH and visualized Western Blot data. FIG. 21A): The GAPDH-normalized signals from the western blot (WB) are plotted on a linear scale. FIG. 21B) The normalized CD24 and CD44 signals net change [TGF-beta (+)—TGF-beta (−)]. FIG. 21C) Summary table of WB signal analysis. The table includes the data that were calculated and converted from the signals of WB analysis as the data-set to be compatible, when the cells did not include TGF-beta as base-line set of “1”, and the higher numbers (bolded text) would be up-regulation, and the numbers below the “1” (base-line) were considered down-regulation (italics texts).

FIG. 22A-C: qRT-PCR analysis of PCBP1 transcripts in cancer stem cells after treatment with PCBP1-GT. Fold change in PCBP1 mRNA expression in U87-MG (CSC) (FIG. 22A) , DU-145 (CSC) (FIG. 22B), and MCF-7 (CSC) (FIG. 22C) cells transfected with the indicated constructs. Dotted lines on y-axis mark 1.5 fold difference, typically indicating level considered for statistical significance. Statistical analysis of molecular assays quantification, was analyzed using the two-tailed Student's t test by RStudio or JMP. For all the significance were defined as p<0.05.

FIG. 23A-C: Western blot analysis of CD133, CD44 and CD24 in DU-145, MCF-7, U87-MG transfected with indicated gene constructs. FIG. 23A: U87-MG (CSC); FIG. 23B: DU-145 (CSC); FIG. 23C: MCF-7 (CSC).

FIG. 24A-C: Measurement of CD44 and CD133 by Western Blot. The western blot signals were normalized against beta actin. FIG. 24A shows CD44 expression. FIG. 24B shows CD133 expression. Summary table (FIG. 24C) shows data that with signals normalized against cells transfected with LV-GFP. Increased levels of expression are shown in bold, and lower levels of expression in italics.

FIG. 25: Niclosamide targets NF-kB and elevates reactive oxygen species (ROS) levels. Niclosamide blocks the TNFα-induced IB phosphorylation, translocation of p65, and the expression of NF-kB regulated genes. Conversely, niclosamide elevates the intracellular ROS content.

FIG. 26: Western blot analysis of U87-MG (CSC) treated with niclosamide (1 μM, 2 μM, 5 μM and 10 μM). Cells were incubated with indicated concentrations of niclosamide for 24 hours (equal volume of DMSO served as vehicle control).

FIG. 27A-C: FIG. 27A) c-Myc signals, normalized against beta actin, are plotted on linear scale. FIG. 27B) Normalized c-Myc signals plotted as net change (i.e., with DMSO signal subtracted). FIG. 27C) Summary table of WB signal analysis, with upregulation shown in bold (>0), and downregulation shown in italics.

FIG. 28A-B: qRT-PCR quantification of c-Myc and BCL-2 expression in U87-MG(CSC) cells treated with DMSA or 10 μM niclosamide. For calculations: DMSO served as base-line in FIG. 28A. Mock-transfection served as base-line in FIG. 28B. Dotted line marks 1.5 fold change in y-axis, considered the benchmark for statistical significance.

FIG. 29A-B: qRT-PCR quantification of c-Myc and BCL-2 expression in DU-145 (CSC) cells treated with DMSA or 10 μM niclosamide. For calculations: DMSO served as base-line in FIG. 29A. Mock-transfection served as base-line in FIG. 29B. Dotted line marks 1.5 fold change in y-axis, considered the benchmark for statistical significance.

FIG. 30A-B. qRT-PCR quantification of c-Myc and BCL-2 expression in DU-145 (CSC) cells treated with DMSA or 10 μM niclosamide. For calculations: DMSO served as base-line in FIG. 30A. Mock-transfection served as base-line in FIG. 30B. Dotted line marks 1.5 fold change in y-axis, considered the benchmark for statistical significance.

### DETAILED DESCRIPTION

Age-related macular degeneration (AMD) is one of the major causes of irreversible blindness in the elderly. Although management of neovascular AMD (wet AMD) has dramatically progressed, there is still no effective treatment for diseases such as non-neovascular AMD (atrophic, or dry AMD) and autoimmune diseases such as Sjögren's syndrome with serious dry eye.

Clinical trials of stem cell therapies have shown promising prospects for retinal pigment epithelial (RPE) cell transplantation. For example, in 2011, Advanced Cell Technology (Santa Monica, Calif., USA) performed Phase I/II clinical trials to elucidate the efficacy of hESC-derived RPE cell transplantation in dry AMD and Stargardt's disease patients. Subsequently, Schwartz et al. (2016) published the results of two Phase I/II trials of 18 patients with dry AMD or Stargardt's disease. After four years, more than half of treated patients experienced sustained improvements in visual acuity and demonstrated evidence of possible cellular engraftment. However, key issues including implantation techniques, immune rejection, and xeno-free components still need to be further investigated and improved. Further, use of human or animal-derived stem cells components that may carry factors such as sialic acid or Neu5Gc, which may cause unwanted immunogenicity of the cells or even expose the patient to certain pathogens, needs to be considered. Therefore, stem cell therapy by iPSC technology and retinal microenvironmental regulation of gene expression represents potential new approaches for dry AMD treatments.

Techniques for generating iPSCs provide a new opportunity to directly induce host (or patient) differentiated tissue into stem cell-like cells without the exogenous administration of stem cell inducing proteins such as Oct4, Sox2, cMyc, or Klf4. While these transcription factors are known to play a role in inducing iPSCs, they may also trigger oncogenesis. Thus, the compositions and methods disclosed herein include cell transduction with an anti-oncogene such as PCBP1 to reduce or prevent oncogenesis.

Without being bound by theory, PCBP1 may take the place of c-Myc in the vectors of the present disclosure as PCBP1 itself may play a role in transforming differentiated cells into stem cells. PCBP1 may transform cells into stem cells either in conjunction with other transcription factors, or on its own. Importantly, PCBP1 does not exhibit the same oncogenesis observed with c-Myc.

These two advanced features (e.g. stem cell-inducing agents or transcription factors involved in the maintenance of stem cell development and PCBP1) are incorporated into a vector system for gene therapy to directly induce conversion of cells into stem cell-like cells for the treatment of disease. These features provide a minimal potential for involving tumorigenic pathways that may be otherwise activated during stem cell development.

### Polypyrimidine Tract-Binding Protein 1 (PCBP1)

In some aspects, the present disclosure provides a vector containing the transcription factor Poly(rC)-binding protein 1 (PCBP1, also known as hnRNP-E1 and αCP1) which can inhibit or delay tumorigenesis. In some embodiments, the PCBP1 is a mammalian PCBP1. In some embodiments, the PCBP1 is human PCBP1, fragment, or variant thereof.

In some embodiments, PCBP1 alters the expression of one or more cancer biomarkers. In some embodiments, the cancer biomarker is a breast cancer biomarker. In some embodiments, the cancer biomarker is a prostate cancer biomarker. In some embodiments, the cancer biomarker is a lung cancer biomarker. In some embodiments, the expression of one or more cancer biomarkers is increased. In some embodiments, the expression of one or more cancer biomarkers is decreased. In some embodiments, the cancer biomarker is associated with metastasis. In some embodiments, the cancer biomarker includes, but is not limited to, PRL-3, STAT3, CD44 variant, CD133, CD24, Estrogen receptor, progesterone receptor, HER2, CA27, CA29, CA25, CEA, CA125, Ki-67, ERα, Cyclin D1, Cyclin E, ERβ, PSA, PCA3, AR-V7, E-cadherin, and vimentin. In some embodiments, transfection of a cancer cell with the constructs of the present disclosure decreases expression of CD44. In some embodiments, transfection of a cancer cell with the constructs of the present disclosure decreases expression of CD133. In some embodiments, the PCBP1 is a nucleic acid. In some embodiments, the nucleic acid is DNA. In some embodiments, the nucleic acid is RNA. In some embodiments, the nucleic acid is mRNA. In some embodiments, the PCBP1 contains the nucleic acid sequence of SEQ ID NO: 1. In some embodiments, the PCBP1 is the full-length nucleic acid sequence of SEQ ID NO: 1. In some embodiments, the PCBP1 is a fragment of the nucleic acid sequence of SEQ ID NO: 1. In some embodiments, the PCBP1 is a variant of the nucleic acid sequence of SEQ ID NO: 1. Without being bound by theory, each of the KH domains within PCBP1 has been shown to bind to mRNAs of different proteins, and use of one or more KH domain may selectively inhibit the translation of a given protein. In some embodiments, the PCBP1 nucleic acid encodes all three K-homologous (KH) domains. In some embodiments, the PCPB1 nucleic acid encodes two KH domains. In some embodiments, the PCPB1 nucleic acid encodes one KH domain.

Further, mutations in a nuclear localization signal may alter the ability of PCBP1 to translocate into the nucleus, and thus affect later gene expression. In some embodiments, the PCBP1 contains a mutation in one or both nuclear localization signals. In some embodiments, the change in one or both nuclear localization signals inhibits the ability of PCBP1 to translocate into the nucleus.

Phosphorylation also plays a role in the activity of PCBP1 (e.g. unphosphorylated PCBP1 may lack activity). In some embodiments, the PCBP1 nucleotide sequence contains mutations that affect the ability of the PCBP1 polypeptide to be phosphorylated. In some embodiments, the PCBP1 nucleotide sequence prevents PCBP1 polypeptide phosphorylation. In some embodiments, the unphosphorylated PCBP1 or not fully phosphorylated PCBP1 polypeptide is not active at wild type levels.

In some embodiments, the PCBP1 nucleotide sequence contains a point mutation relative to wild type PCBP1 (SEQ ID NO: 1). In some embodiments, the PCPB1 nucleotide sequence contains a point mutation that affects phosphorylation. In some embodiments, the PCBP1 nucleotide sequence contains a mutation(s) that may affect nuclear membrane translocation. In some embodiments, the point mutation is selected from the group including, but not limited to, G13A, T299C, T299A, G326A, G527A, C652T, C688T, G781T, G814T, C947T, G1033C, C1034T, or G1048C.

In some embodiments, the disclosure provides mimetics, analogs, derivatives, variants, or mutants of PCBP1 (SEQ ID NO: 1). In some embodiments, the mimetic, analog, derivative, variant, or mutant contains one or more nucleic acid substitutions compared to the nucleic acid sequence of the native PCBP1. In some embodiments, one to 20 nucleic acids are substituted. In some embodiments, the mimetic, analog, derivative, variant, or mutant contains about 1, about 2, about 3, about 4, about 5, about 6, about 7, about 8, about 9, or about 10 nucleic acid substitutions compared to the nucleic acid sequence of the native PCBP1 (e.g. SEQ ID NO: 1). In some embodiments, the mimetic, analog, derivative, variant, or mutant contains one or more nucleic acid deletions compared to the amino acid sequence of the native PCBP1 (e.g. SEQ ID NO: 1).

In some embodiments, one to 20 nucleic acids are deleted compared to the nucleic acid sequence of the native PCBP1 (e.g. SEQ ID NO: 1). In some embodiments, the mimetic, analog, derivative, variant, or mutant has about 1, about 2, about 3, about 4, about 5, about 6, about 7, about 8, about 9, or about 10 nucleic acid deletions compared to the nucleic acid sequence of the native PCBP1 (e.g. SEQ ID NO: 1). In some embodiments, one to ten nucleic acids are deleted at either terminus compared to the nucleic acid sequence of the native PCBP1 (e.g. SEQ ID NO: 1). In some embodiments, one to ten nucleic acids are deleted from both termini compared to the nucleic acid sequence of the native PCBP1. In some embodiments, the nucleic acid sequence of the mimetic, analog, derivative, variant, or mutant is about 70% identical to about 99.9% identical to the nucleic acid sequence of the native PCBP1. In some embodiments, the nucleic acid sequence of the mimetic, analog, derivative, variant, or mutant is at least about 70% identical to the nucleic acid sequence of the native PCBP1. In some embodiments, the nucleic acid sequence of the mimetic, analog, derivative, variant, or mutant is about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90% about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identical to the nucleic acid sequence of the native PCBP1 (e.g. SEQ ID NO: 1). Percentage identity can be calculated using the alignment program EMBOSS Needle, available at http://www.ebi.ac.uk/Tools/psa/emboss_needle/.

In some embodiments, the PCBP1 is a polypeptide. In some embodiments, the PCBP1 contains the amino acid sequence of SEQ ID NO: 2. In some embodiments, the PCBP1 is the full-length amino acid sequence of SEQ ID NO: 2. In some embodiments, the PCBP1 is a fragment of the amino acid sequence of SEQ ID NO: 2. In some embodiments, the PCB1 is a variant of the amino acid sequence of SEQ ID NO: 2. In some embodiments, the PCBP1 is a functional fragment or variant of the amino acid sequence of SEQ ID NO: 2. In some embodiments, the PCBP1 polypeptide contains all three K-homologous (KH) domains. In some embodiments, the PCPB1 polypeptide contains two KH domains. In some embodiments, the PCPB1 polypeptide contains one KH domain.

In some embodiments, the PCBP1 polypeptide sequence contains an amino acid mutation relative to wild type PCBP1 (SEQ ID NO: 2). In some embodiments, the PCPB1 polypeptide sequence contains an amino acid mutation that affects phosphorylation. In some embodiments, the amino acid mutation is selected from the group including, but not limited to, V5M, L100P, L100Q, C109Y, G176E, P218S, S223L, D261Y, A272S, A316V, A345P, A345V, or E350Q.

In some embodiments, the disclosure provides mimetics, analogs, derivatives, variants, or mutants of PCBP1 (SEQ ID NO:2). In some embodiments, the mimetic, analog, derivative, variant, or mutant contains one or more amino acid substitutions compared to the amino acid sequence of the native PCBP1. In some embodiments, one to 20 amino acids are substituted. In some embodiments, the mimetic, analog, derivative, variant, or mutant contains about 1, about 2, about 3, about 4, about 5, about 6, about 7, about 8, about 9, or about 10 amino acid substitutions compared to the amino acid sequence of the native PCBP1 (SEQ ID NO:2).

In some embodiments, the mimetic, analog, derivative, variant, or mutant contains one or more amino acid deletions compared to the amino acid sequence of the native therapeutic peptide agent. In some embodiments, one to 20 amino acids are deleted compared to the amino acid sequence of the native protein agent. In some embodiments, the mimetic, analog, derivative, variant, or mutant has about 1, about 2, about 3, about 4, about 5, about 6, about 7, about 8, about 9, or about 10 amino acid deletions compared to the amino acid sequence of the native PCBP1 (SEQ ID NO:2). In some embodiments, one to ten amino acids are deleted at either terminus compared to the amino acid sequence of the native PCBP1 (SEQ ID NO:2). In some embodiments, one to ten amino acids are deleted from both termini compared to the amino acid sequence of the native PCBP1 (SEQ ID NO:2). In some embodiments, the amino acid sequence of the mimetic, analog, derivative, variant, or mutant is at least about 70% identical to the amino acid sequence of the native PCBP1 (SEQ ID NO:2). In some embodiments, the amino acid sequence of the mimetic, analog, derivative, variant, or mutant is about 70% to about 99.9% identical to the amino acid sequence of native PCBP1 (SEQ ID NO: 2). In some embodiments, the amino acid sequence of the mimetic, analog, derivative, variant, or mutant is about 70%, about 71%, about 72%, about 73%, about 74%, about 75%, about 76%, about 77%, about 78%, about 79% about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identical to the amino acid sequence of the native PCBP1 (SEQ ID NO:2). In some embodiments, the amino acid sequence of the mimetic, analog, derivative, variant, or mutant is about 70%, about 71%, about 72%, about 73%, about 74%, about 75%, about 76%, about 77%, about 78%, about 79% about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9%identical to the amino acid sequence of the native PCBP1 (SEQ ID NO:2) and retains all or most of the biological activity of the native PCBP1. In some embodiments, the amino acid sequence of the mimetic, analog, derivative, variant, or mutant is about 70%, about 71%, about 72%, about 73%, about 74%, about 75%, about 76%, about 77%, about 78%, about 79% about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9%identical to the amino acid sequence of the native PCBP1 (SEQ ID NO:2) and has reduced or altered activity compared with the native PCBP1.

In some embodiments, the amino acid sequence of the mimetic, analog, derivative, variant, or mutant is about 70% to about 99.9% identical to the amino acid sequence or of one or more domains of the native PCBP1 (SEQ ID NO: 2). In some embodiments, the amino acid sequence of the mimetic, analog, derivative, variant, or mutant is about 70%, about 71%, about 72%, about 73%, about 74%, about 75%, about 76%, about 77%, about 78%, about 79% about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identical to the amino acid sequence of one or more domains of the native PCBP1 (SEQ ID NO:2). In some embodiments, the amino acid sequence of the mimetic, analog, derivative, variant, or mutant is about 70% to about 99.9% identical to the amino acid sequence or of one or more domains of the native PCBP1 (SEQ ID NO: 2) and retains all or most of the biological activity of the native PCBP1. In some embodiments, the amino acid sequence of the mimetic, analog, derivative, variant, or mutant is about 70%, about 71%, about 72%, about 73%, about 74%, about 75%, about 76%, about 77%, about 78%, about 79% about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9%identical to the amino acid sequence of one or more domains of the native PCBP1 (SEQ ID NO:2) and retains all or most of the biological activity of the native PCBP1. In some embodiments, the amino acid sequence of the mimetic, analog, derivative, variant, or mutant is about 70% to about 99.9% identical to the amino acid sequence or of one or more domains of the native PCBP1 (SEQ ID NO: 2) and has reduced or altered activity compared to the native PCBP1. In some embodiments, the amino acid sequence of the mimetic, analog, derivative, variant, or mutant is about 70%, about 71%, about 72%, about 73%, about 74%, about 75%, about 76%, about 77%, about 78%, about 79% about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identical to the amino acid sequence of one or more domains of the native PCBP1 (SEQ ID NO:2) and has reduced or altered activity compared with the native PCBP1. Percentage identity can be calculated using the alignment program EMBOSS Needle, available at http://www.ebi.ac.uk/Tools/psa/emboss_needle/. The following default parameters may be used for Pairwise alignment: Protein Weight Matrix=BLOSUM62; Gap Open=10; Gap Extension=0.1.

### Transcription Factors

Transcription factors promote the differentiation of embryonic stem (ES) cells derived from the inner cell mass of the blastocyst into stem or progenitor cells of all three vertebrate germ layers: ectoderm, mesoderm, and endoderm. In addition, the expression of specific transcription factors is sufficient to reprogram somatic cells back to a pluripotent state. Induced pluripotent stem (iPSC) cells, like ES cells, give rise to differentiated stem cells such as neural and epithelial stem cells. Differentiated stem cells function to replenish cells of the tissue in which they are found.

In some embodiments, the one or more additional transcription factors are known to be associated with stem cell induction. In some embodiments, the one or more additional transcription factors are known to be associated with stem cell renewal and/or pluripotency. In some embodiments, the one or more additional transcription factors associated with stem cell renewal and/or pluripotency are selected from, but not limited to, Brachyury, FoxD3, GBX2, Max, NFkB1, Pax6, c-Jun, c-Maf, c-Myc, FoxF1, Goosecoid, Mef2c, NFkB2, PRDM14, FoxH1, HES-1, MIXL1, Oct-3/4, Rex-1/ZFP42, FoxO1/FKHR, HNF-3 alpha/FoxA1, MTF2, OCT4, SALL1, C/EBP alpha, GATA-2, KLF2, Nanog, Otx2, SALL4, EOMES, GATA-3, KLF4, NFkB/IkB Activators, p53, Smad1, FoxC2, Gata4, KLF5, NFkB/IkB Inhibitors, Pax2, Smad1/5, Smad2, Smad 2/3, Smad 3, Smad 4, Smad 5, Smad 8, Snail, SOX15, SUZ12, UTF1, SOX17, SEMA 6A-1, WDR5, SOX2, SFRP1, WT1, SOX7, Tbx5, ZNF206, STAT Activators, TBX6, ZNF281, STAT Inhibitors, TCF-3/E2A, Snail, STAT3, and THAP11.

In some embodiments, the transcription factor is involved in one or more oncogenesis pathways. In some embodiments, overexpression of the transcription factor is involved in one or more oncogenesis pathways (e.g. SOX2 overexpression has been described in lung cancer tissues). In some embodiments, the expression changes of the transcription factor are biomarkers of cancer. In some embodiments, the cancer is liver cancer or lung cancer.

Any appropriate transcription factors may be used, including but not limited to ASCL2/Mash2, ASCL1/Mash1, CDX2, DNMT1, ELF3, Ets-1, FoxM1, FoxN1, Hairless, HNF-4, alpha/NR2A1, IRF6, MITF, Miz-1/ZBTB17, MSX1, MSX2, MYB, Neurogenin-3, NFATC1, NKX3.1, Nrf2, p53, p63/TP73L, Pax2, Pax3, Pax4, Pax6, RUNX1/CBFA2, RUNX2/CBFA1, RUNX3/CBFA3, Smad1, Smad1/5, Smad2, Smad2/3, Smad3, Smad4, Smad5, Smad7, Smad8, Smad9, Snail, SOX9, STAT Activators, STAT Inhibitors, STAT1, STAT3, STAT4, STAT5a, STAT6, SUZ12, TCF-3/E2A, TCF7/TCF1, Brachyury, GATA-1, GATA-2, GATA-3, GATA-4, GATA-6, LMO2, LMO4, SCL/Tal1, AHR, Aiolos/IKZF3, CDX4, CREB, DNMT3A, DNMT3B, EGR1, FoxO3, Helios, HES-1, HHEX, HIF-1 alpha, HMGB1/HMG-1, HMGB3, Ikaros, c-Jun, LMO2, LMO4, c-Maf, MafB, MEF2C, MYB, NFATC2, NFIL3/E4BP4, PITX2, PRDM16/MEL1, Prox1, PU.1/Spi-1, SALL4, SCL/Tal1, Spi-B, TSC22, HNF-3 alpha/FoxA1, HNF-3 beta/FoxA2, ONECUT2/OC-2, TBX3, TBX5, TBX18, TCF-2/HNF-1 beta, PDX-1/IPF1, PTF1A, RFX6, EBF-1, EBF-2, EBF-3, ETV5, FoxC2, FoxF1, HMGA2, MYF-5, Myocardin, MyoD, Myogenin, PLZF, Twist-1, Twist-2, ATF2, Brg1, CSL, EMX2, FosB/G0S3, GLI-1, GLI-2, HOXB1, MCM2, MCM7, NeuroD1, NeuroD2, Neurogenin-1, Neurogenin-2, NKX2.2, NKX6.1, Olig1, Olig2, Olig3, RCOR1/CoREST, SOX1, SOX2, SOX3, SOX5, SOX6, TCF-12/HTF4, ZIC1, ZIC3, EOMES, FoxD3, FoxH1, FoxO1/FKHR, GBX2, Goosecoid, KLF2, KLF4, KLF5, Max, MIXL1, MTF2, NFkB/IkB Activators, NFkB/IkB Inhibitors, NFkB1, NFkB2, Oct-3/4, Otx2, PRDM14, Rex-1/ZFP42, SALL1, SOX7, SOX15, SOX17, SOX18, TBX6, THAP11, UTF1, WDR5, WT1, ZNF206, ZNF281, ATBF1/ZFHX3, HIPK1, HIPK2, PIAS2, PIAS3, TRIM21, TRIM32, WTX, ZMIZ1/Zimp10, Carm1, CBP, Cbx2, CHD1, CHD7, CTCF, DEK, MBD3, NCOA2, NCOA3PIWIL1/HIWI, PIWIL2, PIWIL4, RBBP4, SATB1, SIN3A, SMARCA5/SNF2H, TAF5L, ARA54, ATAD2, beta-Catenin, Bcl-9, BTF3, CBFB, Cyclin A1, Cyclin A2, Cyclin B1, Cyclin B2, Cyclin D3, DDX17, DDX5, IKK alpha, IKK beta, LEDGF, LITAF, LXR alpha/NR1H3, MED4, Park7/DJ-1, PGC1 alpha, Pygopus-1, Pygopus-2, RNF4, TACC3, Tankyrase 1, Tankyrase Inhibitors, TAZ/WWTR1, TORC1, TORC2, TORC3, TRRAP, YAP1, ACLP, ATN1, BASP1, Bcl-6, CDP/CUTL1, CIS-1, Cited-2, COMMD1, CREG, CtBP1, DEPTOR/DEPDC6, FHL1, FHL2, FIH-1/HIF-1AN, GFI-1, Host Cell Factor 1/HCFC1, Hexim 1, Histone Deacetylase 2/HDAC2, Histone Deacetylase 8/HDAC8, ID1, ID2, IkB-alpha, IkB-beta, IkB-epsilon, IRF2BP1, KAP1, Keap1, LCoR, NCOR1, NCOR2, p15INK4b/CDKN2B, p16INK4a/CDKN2A, p18INK4c/CDKN2C, PA2G4, Prohibitin 2, SMURF2, SOCS-1, SOCS-2, SOCS-3, SOCS-4, SOCS-5, SOCS-6, SOCS-7/Nck/NAP4, TANK, TLE1, TLE2, TLE3, TMEM18, TMEM87A, ZBTB38, ZBTB7A/Pokemon, ZFP90Oct4, Sox 2, Nodal, FGF, TGF-β, LIF, BMP4, KLF4, KLF2, Myc, LIN28A, TUBB, HAP90AB1, PELI1, SEMA6A, SLC7A5, RGMB, TUBB6, HSPA4, SFRP1, LRRN1, PRDM14, C/EBP alpha, EOMES, and Nanog, or fragments thereof. In some embodiments, the one or more additional transcription factors are SOX2, Oct4, and/or Klf4 or fragments or variants thereof.

### Vectors and Plasmids

Efficient delivery of the therapeutic gene to the target tissue or cell is the most significant hurdle for successful gene therapy. Since naked DNA is rapidly cleared or degraded in vivo by phagocytic immune cells or extracellular nucleases, a means of protecting the transgene may be desired. Furthermore, a vehicle for effecting tissue or cell entry is also required, due to the poor efficiency of spontaneous DNA uptake. Thus, DNA is normally combined with a gene delivery vehicle of some type, commonly known as a vector, to protect and mediate effective tissue or cell entry of the gene of interest.

Gene delivery systems can be grouped into non-biological (e.g. chemical and physical approaches of introducing plasmid DNA to mammalian cells) or biological (e.g. viruses and bacteria). Non-viral gene delivery systems normally involve the transfer genes carried on plasmid DNA. Plasmids employed do not generally replicate in mammalian cells.

Most commonly, recombinant viruses or naked DNA or DNA complexes are used. For example, viruses can be modified in the laboratory to provide vectors that carry corrected, therapeutic DNA into cells, where it can be integrated into the genome to alter abnormal gene expression and correct genetic disease. Alternatively, the vector may remain extrachromosomal and be expressed transiently.

In some aspects, the present disclosure provides methods of gene therapy using viral vectors. Viruses that may be used in gene therapy include, but are not limited to, lentiviruses, retroviruses, adenoviruses, adeno-associated viruses, replication-competent vectors, vaccinia virus, and the herpes simplex virus.

In some aspects, the present disclosure provides methods of gene therapy using non-viral vectors. In some aspects, the present disclosure provides methods of gene therapy using bacterial vectors. In some embodiments, the gene therapy method involves injection of naked nucleic acids (e.g. DNA or RNA). This may be performed using any appropriate means known in the art.

In some aspects, the present disclosure provides methods of gene therapy using non-viral and non-bacterial vectors. In some embodiments, the non-viral and non-bacterial vector is a eukaryotic vector. In some embodiments the methods of gene therapy provide CRISPR, TALEN, or zinc finger proteins (ZFNs). In some embodiments, the eukaryotic vector includes a transposon system. In some embodiments, the transposon system is the Tn5, CRISPR-associated, Sleeping Beauty, or piggyBac transposon systems.

In some aspects, the present disclosure provides methods of gene therapy using nanoparticles. In some embodiments, the nanoparticle is a lipid-based nanoparticle. In some embodiments, the lipid-based nanoparticle is a solid lipid-nanoparticle (SLN). In some embodiments, the lipid-based nanoparticle is an unstructured lipid carrier (NLC). In some embodiments, the nanoparticle is a polymer-based nanoparticle. In some embodiments, the polymer-based nanoparticle is a nanosphere or a nanocapsule.

Plasmid DNA-based vectors are commonly used in gene therapy and can accommodate large segments of DNA and allows the manipulation of a variety of regulatory elements that impact gene transfer and expression. At its most basic, an expression plasmid contains an expression cassette and backbone. The expression cassette is a transcriptional unit containing the gene or genes of interest and any regulatory sequences required for expression in the target cells. The backbone may contain a selectable marker (e.g. an antibiotic resistance gene or an auxotrophic selection gene) and/or an origin of replication required for the production of the plasmid in bacteria. In some embodiments, the plasmid does not contain a selectable marker or traditional replication component (e.g. minicircles, self-replicating minicircles, ministrings, linear DNAs, etc.).

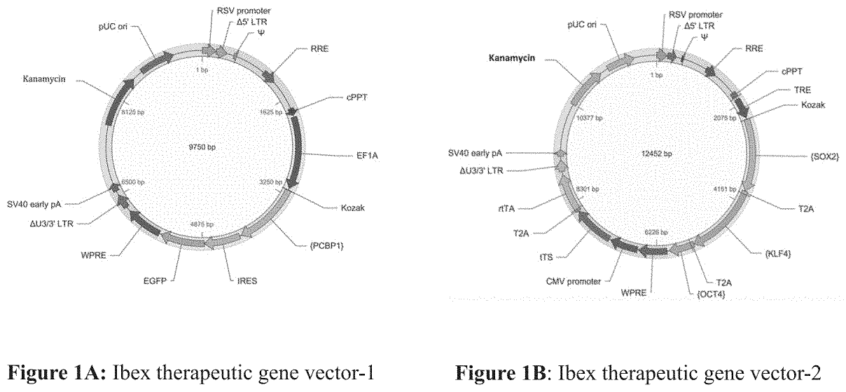

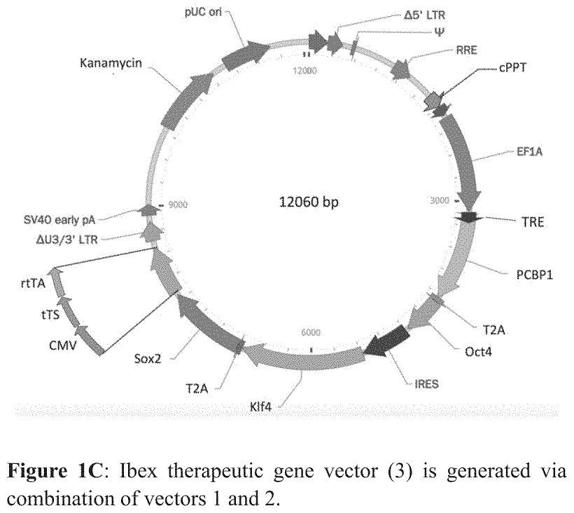

Any appropriate plasmid may be used in the methods disclosed herein. In some embodiments, representative plasmids are disclosed in FIG. 1.

Any appropriate promoter may be used to drive expression of the genes in the expression cassette. Regulated and/or inducible promoters are important as they allow the induction of stem cells in situ according to different conditions that may be required in clinical applications. These promoters may be able to prevent stem cells from reproducing/being induced indefinitely and growing non-stop.

In some embodiments, the plasmid contains a retroviral promoter. In some embodiments, the plasmid contains a viral promoter. In some embodiments, the plasmid contains a mammalian promoter. In some embodiments, the mammalian promoter is a human promoter. In some embodiments, the promoter is tissue or cell specific. In some embodiments, the tissue specific promoter is selected from those listed in Table 1. In some embodiments, the promoter is specific for retinal tissue or cells. In some embodiments, the promoter is specific for hematopoietic cells. In some embodiments, the promoter is specific for liver tissue or cells. In some embodiments, the promoter is specific for lung tissue or cells. In some embodiments, the promoter is specific for muscle tissue or cells. In some embodiments, the promoter is specific for HIV-infected cells.

TABLE 1
Tissue Specific Promoters

| Promoter | Note |
|---|---|
| GANT2 (Guanine nucleotide- | Used with IRBP (interphotoreceptor |
| binding protein G(t) subunit | retinoid binding protein) |
| alpha-2) | |
| VMD2 (vitelliform macular | 3.0 kb, use VMD2 promoter direct |
| dystrophy) | Tetracycline inducible system, |
| | controlled Cre expression in transgenic |
| | mice |
| Human IRBP (interphotoreceptor | Sequence: ATTCTCATCGTAAATCAGGCTC |
| retinoid binding protein) | ACTTCCATTG GCTGCATACG GTGGAGTGAT |
| | GTGACCATAT GTCACTTGAGCATTACACAA |
| | ATCCTAATGA GCTAAAAATA TGTTTGTTTT |
| | AGCTAATTGA CCTCTTTGGCCTTCATAAAG |
| | CAGTTGGTAA ACATCCTCAG ATAATGATTT |
| | CCAAAGAGCA GATTGTGGGTCTCACCTGTG |
| | CAGAGAAAGC CCACGTCCCT GAGACCACCT |
| | TCTCCAG~G CCTACTGAGGCACACAGGGG |
| | CGCCTGCCTG CTGCCCGCTC AGCCAAGGCG |
| | GTGTTGCTGGA (SEQ ID NO: 3) |
| Tetracycline | Sequence: |
| | GGTACCGAGCTCGACTTTCACTTTTCTCTATCAC |
| | TGATAGGGAGTGGTAAACTCGACTTTCACTTTT |
| | CTCTATCACTGATAGGGAGTGGTAAACTCGACT |
| | TTCACTTTTCTCTATCACTGATAGGGAGTGGTAA |
| | ACTCGACTTTCACTTTTCTCTATCACTGATAGGG |
| | AGTGGTAAACTCGACTTTCACTTTTCTCTATCAC |
| | TGATAGGGAGTGGTAAACTCGACTTTCACTTTT |
| | CTCTATCACTGATAGGGAGTGGTAAACTCGACT |
| | TTCACTTTTCTCTATCACTGATAGGGAGTGGTAA |
| | ACTCGACCTATATAAGCAGAGCTCGTTTAGTGA |
| | ACCGTCAGATCGCCTGGAGACGCCATCCACGCT |
| | GTTTTGACCTCCATAGAAGACACCGGGACCGAT |
| | CCAGCCTCCGCGGCCCCGAATTG (SEQ ID NO: 4) |

In some embodiments, the promoter is an inducible promoter. In some embodiments, the inducible promoter is selected from those listed in Table 2. In some embodiments, the promoter is constitutive. In some embodiments, the promoter is a synthetic promoter or contains enhancer elements. In some embodiments, the promoter is a hybrid promoter. In some embodiments, the hybrid promoter contains regulatory regions of a gene. In some embodiments, the promoter is selected from the group including, but not limited to, Ef1a, CAG, sv40, CMV, RSV, Oct4, Rex1, Nanog, GANT2, VMD2, hIRBP, TET promoter, CAAT Box, CG Box, GT-1 motif, I-box, AT-rich sequence, RBCS1, TRE3G, GAL1, Lap267, Rapamycin, CD11a, CD11b, CD18, Beta-globin promoter/LCR, Immunoglobulin promoter, PEPCK promoter, Albumin promoter, hAAT, SPC, SP-A, MCK, VLC1, HIV-LTR, tTS (tetracycline transcription silencer), Tat/Rev-responsive elements, Tat-inducible element, and FMR1.

TABLE 2
Inducible Promoters

| Promoter | Note |
|---|---|
| TRE3G | Gene therapy use: will express high level of GOI (gene |
| | of interest) only when have doxycycline |
| Tetracycline | A TRE is 7 repeats of a 19 nucleotide tetracycline |
| | operator (tetO) sequence, and is recognized by the |
| | tetracycline repressor (tetR). In the endogenous |
| | bacterial system, if tetracycline, or one of its |
| | analogs like doxycycline, are present, tetR will bind |
| | to tetracycline and not to the TRE, permitting |
| | transcription. |
| Lac promoter | Expressed in bacteria cell line, induced by IPTG |
| Ecdysone | Can be expressed in mammalian cell line |
| Rapamycin | AAV vectors were co-infused, one expressing the |
| | transcription factors and one encoding the hAADC |
| | transgene downstream from a rapamycin-inducible |
| | promoter. |

In some embodiments, the plasmid contains all of the genes to be introduced via gene therapy. In some embodiments, the plasmid contains an anti-oncogene. In some embodiments, the plasmid contains an anti-oncogene and one or more transcription factors.

In some embodiments, the methods disclosed herein introduce all the desired genes on one plasmid. In some embodiments, the methods disclosed herein employ one or more plasmids. In some embodiments, the anti-oncogene is on one plasmid and the one or more transcription factors are on another plasmid. In some embodiments, the anti-oncogene and one transcription factor are on one plasmid, and one or more transcription factors are on another plasmid. In some embodiments, the anti-oncogene and two or more transcription factors are on one plasmid and one or more transcription factors are on another plasmid. In some embodiments, the anti-oncogene and the other transcription factors are on one plasmid. In some embodiments, the anti-oncogene and three other transcription factors are on one plasmid.

In some embodiments, the anti-oncogene is PCBP1. In some embodiments, the one or more transcription factors are Oct4, Sox2, and/or Klf4.

### Diseases and Disorders

The methods disclosed herein may be employed on any appropriate cell or tissue type. In some embodiments, the methods disclosed herein are performed in vitro. In some embodiments, the methods disclosed herein are performed ex vivo. In some embodiments, the methods disclosed herein are performed on isolated cells. In some embodiments, the methods disclosed herein are performed on cell culture. In some embodiments, the isolated cells or cell culture cells are taken from the subject and then implanted after iPSC induction.

In some embodiments, the methods disclosed herein are employed in vivo. In some embodiments, the methods disclosed herein are employed in situ. In some embodiments, the methods disclosed herein are used to induce iPSCs in any appropriate part of the body, including, but not limited to, the eye, retina, heart, blood, white blood cell, red blood cell, platelet, vitreous humor, sclera, retina, iris, cornea, skeletal muscle, cardiac muscle, smooth muscle, cartilage, tendon, bone, epidermis, organ, liver, heart, kidney, lung, stomach, gastrointestinal tract, colon, bladder, ovary, testes, pancreas, bone marrow, and/or gland.

The methods disclosed herein may be used to treat, prevent, ameliorate, or delay any appropriate disease. For example, diseases that may be treated, prevented, ameliorated, or delayed are characterized by injury and/or degeneration. In some embodiments, the disease or disorder includes, but is not limited to, macular degeneration, age-related macular degeneration, neovascular AMD (wet AMD), retinitis pigmentosa (RP), Stargardt's disease retinal degeneration, heart attack, myocardial infarction, autoimmune disorders, Systemic Lupus Erythematosus, type 1 diabetes, type 2 diabetes, heart disease, cardiomyopathy, cystic fibrosis, multiple sclerosis, graft-versus-host disease, stroke, Alzheimer's disease, Parkinson's disease, spinal cord injury, arthritis, emphysema, Crohn's disease, organ failure, organ degeneration due to aging and other pathological conditions.

In some aspects, the present disclosure does not comprise compositions or methods treating cancer or inhibiting the growth/migration of cancer cells by introducing PCBP1 alone into a cell. In some embodiments, the present disclosure does not comprise the subject matter of PCT/US2019/016688 filed Feb. 5, 2019.

### Administration

The compositions of the present disclosure may be administered via any appropriate means. In some embodiments, the vector is administered transdermally, via injection, intramuscularly, subcutaneously, orally, nasally, intra-vaginally, rectally, transmucosally, enterally, parenterally, topically, epidurally, intracerebraly, intracerebroventricularly, intraarterially, intraarticularly, intradermaly, intralesionaly intraocularly, intraosseously intraperitonealy, intrathecally, intrauterinely, intravenously, intravesical infusion, or intravitreally.

In some aspects, the disclosure provides one or more additional transcription factors that play a role in stem cell induction. In some embodiments, the anti-oncogene is administered with one additional transcription factor. In some embodiments, the anti-oncogene is administered with two additional transcription factors. In some embodiments, the anti-oncogene is administered with three or more additional transcription factors. In some embodiments, the anti-oncogene is PCBP1. In some embodiments, the one or more transcription factors are Sox2, Oct4, and/or Klf4.

In some embodiments, iPSCs created using the methods disclosed herein are administered to a patient. In some embodiments, iPSCs created using the methods disclosed herein are administered to a patient to treat, prevent, ameliorate, or delay the onset of a disease or disorder. In some embodiments, administration of the vectors or plasmids disclosed herein induce iPSCs in the subject. In some embodiments, administration of the vectors or plasmids disclosed herein induce iPSCs in the subject and treat, prevent, ameliorate, or delay the onset of a disease or disorder. In some embodiments, the disease or disorder is a degenerative disease or disorder.

In some embodiments, the vectors, plasmids, or cells disclosed herein are administered once to a patient. In some embodiments, the vectors, plasmids, or cells disclosed herein are administered about 2 times, about 3 time, about 4 times, about 5 times, about 6 times, about 7 times, about 8 times, about 9 times, about 10 times, about 20 times, about 40 times, or more to a patient. Vectors, plasmids, or cells disclosed herein are administered until disease or disorder symptoms improve.

In some embodiments, administration of the vectors or plasmids disclosed herein induce iPSCs in a treated patient compared to an untreated patient or the same patient before treatment. In some embodiments, the iPSCs are induced in a treated patient between week 1 and year 10. In some embodiments, administration of the plasmids or vectors disclosed herein induces iPSCs at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with iPSC induction in an untreated patient or the same patient before treatment. In some embodiments, administration of the plasmids or vectors disclosed herein induces iPSCs for about 1 week, about 1 month, about 2 months, about 3 months, about 4 months, about 5 months, about 6 months, about 1 year, about 2 years, about 5 years, or about 10 years, or more compared with iPSC induction in an untreated patient or the same patient before treatment.

In some embodiments, the vectors, nucleic acids, or cells disclosed herein reduce cancer biomarker expression in treated cells in vitro. In some embodiments, administration of the vectors, nucleic acids, or cells disclosed herein reduces cancer biomarker expression in a treated patient. In some embodiments, administration of the vectors, nucleic acids, or cells disclosed herein reduce cancer biomarker expression in a treated patient or in vitro cells between day 1 and year 10. In some embodiments, administration of the nucleic acids, vectors, or cells disclosed herein reduces cancer biomarker expression at about day 1, about day 2, about day 3, about day 4, about day 5, about day. In some embodiments, iPSC induction is increased by about 1%, about 5%, about 10%, about 20%, about 30%, about 40%, about 50%, about 60%, about 70%, about 80%, about 90%, or about 100% compared with iPSC induction in an untreated patient or the same patient before treatment. In some embodiments, administration of the plasmids or vectors disclosed herein induces iPSCs by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients or in vitro cells treated with other iPSC induction methods. iPSC induction in an untreated patient or the same patient before treatment. In some embodiments, administration of the plasmids or vectors induces iPSCs by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared with iPSC induction in an untreated patient or the same patient before treatment.

In some embodiments, administration of the vectors, plasmids, or cells disclosed herein reduces cancer biomarker expression in a treated patient. In some embodiments, administration of the vectors, plasmids, or cells disclosed herein reduce cancer biomarker expression in a treated patient between week 1 and year 10. In some embodiments, administration of the plasmids, vectors, or cells disclosed herein reduces cancer biomarker expression at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients treated with other iPSC induction methods. In some embodiments, administration of the plasmids, vectors, or cells disclosed herein reduces cancer biomarker expression for about 1 week, about 1 month, about 2 months, about 3 months, about 4 months, about 5 months, about 6 months, about 1 year, about 2 years, about 5 years, or about 10 years, or more compared with controls or patients treated with other iPSC induction methods.

In some embodiments, cancer biomarker expression is decreased by about 1%, about 5%, about 10%, about 20%, about 30%, about 40%, about 50%, about 60%, about 70%, about 80%, about 90%, or about 100% compared with controls or patients treated with other iPSC induction methods. In some embodiments, administration of the plasmids, vectors, or cells disclosed herein reduces cancer biomarker expression by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients or in vitro cells treated with other iPSC induction methods. In some embodiments, administration of the plasmids, vectors, or cells disclosed herein reduces cancer biomarker expression by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared with controls or patients or in vitro cells treated with other iPSC induction methods. In some embodiments, the other iPSC induction methods do not include PCBP1, a fragment or variant thereof.

In some embodiments, administration of the vectors, plasmids, or cells disclosed herein reduces metastasis or tumorigenesis in a treated patient. In some embodiments, administration of the vectors, plasmids, or cells disclosed herein reduces metastasis or tumorigenesis in a treated patient between week 1 and year 10. In some embodiments, administration of the plasmids, vectors, or cells disclosed herein reduces metastasis or tumorigenesis at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients treated with other iPSC induction methods. In some embodiments, administration of the plasmids, vectors, or cells disclosed herein reduces metastasis or tumorigenesis for about 1 week, about 1 month, about 2 months, about 3 months, about 4 months, about 5 months, about 6 months, about 1 year, about 2 years, about 5 years, or about 10 years, or more compared with controls or patients treated with other iPSC induction methods.

In some embodiments, metastasis or tumorigenesis is decreased by about 1%, about 5%, about 10%, about 20%, about 30%, about 40%, about 50%, about 60%, about 70%, about 80%, about 90%, or about 100% compared with controls or patients treated with other iPSC induction methods. In some embodiments, administration of the plasmids, vectors, or cells disclosed herein reduces metastasis or tumorigenesis expression by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients, or in vitro or ex vivo organ or tissue treated with other iPSC induction methods. In some embodiments, administration of the plasmids, vectors, or cells disclosed herein reduces metastasis or tumorigenesis for about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years, or more compared with controls or patients, or in vitro or ex vivo organ or tissue treated with other iPSC induction methods.

In some embodiments, metastasis or tumorigenesis is decreased by about 1%, about 5%, about 10%, about 20%, about 30%, about 40%, about 50%, about 60%, about 70%, about 80%, about 90%, or about 100% compared with controls or patients treated with other iPSC induction methods. In some embodiments, administration of the nucleic acids, vectors, or cells disclosed herein reduces metastasis or tumorigenesis expression by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared controls or patients or in vitro or ex vivo organ or tissue treated with other iPSC induction methods. In some embodiments, the other iPSC induction methods do not include PCBP1, a fragment or variant thereof.

In some embodiments, administration of the plasmids, vectors, or cells disclosed herein reduce disease or disorder symptoms in a treated patient compared to an untreated patient or the same patient before treatment. In some embodiments, the disease or disorder symptoms are measured in a treated patient between week 1 and year 10. In some embodiments, administration of the plasmids, vectors, or cells disclosed herein reduces a disease or disorder symptom at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the disease or disorder symptom in an untreated patient or the same patient before treatment. In some embodiments, administration of the plasmids, vectors, or cells disclosed herein reduces a disease or disorder symptom for about 1 week, about 1 month, about 2 months, about 3 months, about 4 months, about 5 months, about 6 months, about 1 year, about 2 years, about 5 years, or about 10 years, or more compared with the disease or disorder symptom in an untreated patient or the same patient before treatment.

In some embodiments, the disease or disorder symptom is reduced by about 1%, about 5%, about 10%, about 20%, about 30%, about 40%, about 50%, about 60%, about 70%, about 80%, about 90%, or about 100% compared with the disease or disorder symptom in an untreated patient or the same patient before treatment. In some embodiments, administration of the plasmids, vectors, or cells disclosed herein reduces the disease or disorder symptom by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the disease or disorder symptom in an untreated patient or the same patient before treatment. In some embodiments, administration of the plasmids, vectors, or cells disclosed herein reduces the disease or disorder symptom by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared with the disease or disorder symptom in an untreated patient or the same patient before treatment.

In some embodiments, administration of the vectors or plasmids disclosed herein reduce symptoms of AMD in a treated patient compared to an untreated patient or the same patient before treatment. In some embodiments, the symptom is blurred vision, decrease in visual acuity, partial loss of vision, and/or an inability to see in dim light. In some embodiments, the AMD symptoms are measured in a treated patient between week 1 and year 10. In some embodiments, administration of the plasmids, vectors, or cells disclosed herein reduces an AMD symptom at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the AMD symptom in an untreated patient or the same patient before treatment. In some embodiments, administration of the plasmids, vectors, or cells disclosed herein reduces an AMD symptom for about 1 week, about 1 month, about 2 months, about 3 months, about 4 months, about 5 months, about 6 months, about 1 year, about 2 years, about 5 years, or about 10 years, or more compared with the AMD symptom in an untreated patient or the same patient before treatment.

In some embodiments, the AMD symptom is reduced by about 1%, about 5%, about 10%, about 20%, about 30%, about 40%, about 50%, about 60%, about 70%, about 80%, about 90%, or about 100% compared with the AMD symptom in an untreated patient or the same patient before treatment. In some embodiments, administration of the plasmids, vectors, or cells disclosed herein reduces the AMD symptom by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the AMD symptom in an untreated patient or the same patient before treatment. In some embodiments, administration of the plasmids, vectors, or cells disclosed herein reduces the AMD symptom by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared with the AMD symptom in an untreated patient or the same patient before treatment.

In some embodiments, administration of the vectors or plasmids disclosed herein reduce damage caused by a heart attack in a treated patient compared to an untreated patient or the same patient before treatment. In some embodiments, the damage is increased scar tissue, cell death, or inefficient heart activity. In some embodiments, the heart attack damages are measured in a treated patient between week 1 and year 10. In some embodiments, administration of the plasmids, vectors, or cells disclosed herein reduces heart attack damage at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the heart attack damage in an untreated patient or the same patient before treatment. In some embodiments, administration of the plasmids, vectors, or cells disclosed herein reduces heart attack damage for about 1 week, about 1 month, about 2 months, about 3 months, about 4 months, about 5 months, about 6 months, about 1 year, about 2 years, about 5 years, or about 10 years, or more compared with the heart attack damage in an untreated patient or the same patient before treatment.

In some embodiments, the heart attack damage is reduced by about 1%, about 5%, about 10%, about 20%, about 30%, about 40%, about 50%, about 60%, about 70%, about 80%, about 90%, or about 100% compared with the heart attack damage in an untreated patient or the same patient before treatment. In some embodiments, administration of the plasmids, vectors, or cells disclosed herein reduces the heart attack damage by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the heart attack damage in an untreated patient or the same patient before treatment. In some embodiments, administration of the plasmids, vectors, or cells disclosed herein reduces the heart attack damage by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared with the heart attack damage in an untreated patient or the same patient before treatment.

### Kits

The disclosure also provides kits for iPSC induction. In some embodiments, the kits include a vector or plasmid of the present disclosure. The kit can further include a label or printed instructions instructing the use of described reagents. The kit can further include a treatment to be tested. The kits are applied for in vitro and in vivo iPSC induction.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

“Anti-oncogene” as used herein refers to any gene that prevents, delays, prohibits, or otherwise alters expression or activity of oncogenes. In some embodiments, the anti-oncogene is a tumor suppressing gene. In some embodiments the anti-oncogene encodes a transcription factor or element involved in stem cell development.

It should be understood that singular forms such as “a,” “an,” and “the” are used throughout this application for convenience, however, except where context or an explicit statement indicates otherwise, the singular forms are intended to include the plural. All numerical ranges should be understood to include each and every numerical point within the numerical range, and should be interpreted as reciting each and every numerical point individually. The endpoints of all ranges directed to the same component or property are inclusive, and intended to be independently combinable.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

The term “about”, as used herein to refer to a numerical quantity, includes “exactly” plus or minus up to 10% of that referenced numeric indication. When the term “about” is used in reference to a range of values, the term “about” refers to both the minimum and maximum value of the range (e.g., “about 1-50 μm” means “about 1 μm to about 50 μm”). The term “intimately associated”, as used herein to describe the spatial relationship between two or more components of a composition refers to components that are intimately mixed, such as, for example, in mixtures, coatings and matrices.

All publications, patents, and patent publications cited are incorporated by reference herein in their entirety for all purposes.

This disclosure is further illustrated by the following non-limiting examples.

### EXAMPLES

### Example 1—Role of PCPB1 and Transcription Factors in Inducing Retinal Stem Cells

Retinal degeneration is the deterioration of the retina, caused by the progressive and eventual death of the cells of the retina. These retinopathies affect approximately one in 2000 individuals worldwide.

Currently, stem cell therapies and gene therapies are making headlines for their potential to cure diseases, including those that affect vision, however, these stem cell therapies require ex vivo modifications prior to infusion or implantation in order to make a successful therapeutic product. This is due to a number of reasons, principal among them, the need to deliver molecules that reprogram cell gene expression patterns and induce pluripotent stem cells (iPSCs). While this is typically done in the laboratory on isolated cells, disclosed herein is an approach for generating iPSCs in situ, e.g., directly in the eye.

Construction of gene therapy vector system for treatment on retinal degenerative disease: All genes involved in the generation of iPSCs were inserted into one vector and transduced into retinal cells. FIG. 1 shows a vector system that contains the strong human elongation factor-1 alpha (EF) promoter to drive co-expression of genes for PCBP1, GFP (FIG. 1A), as well as the genes for the transcription factors SOX2, Oct4, and KLF4 (FIG. 1B). The final product is a vector containing all cassettes for these transcriptional factors in a single delivery vector system (FIG. 1C). Turn-on of each therapeutic gene is driven by the promoter(s) under the control or inducible manner.

Results: In vitro therapeutic efficacy of induction of differentiated retinal cells into stem cell-like cells: A differentiated retinal cell culture line (ARPE-19) was obtained from ATCC™, and grown in medium without FBS. The cell culture was subsequently split for gene transfection and infection.

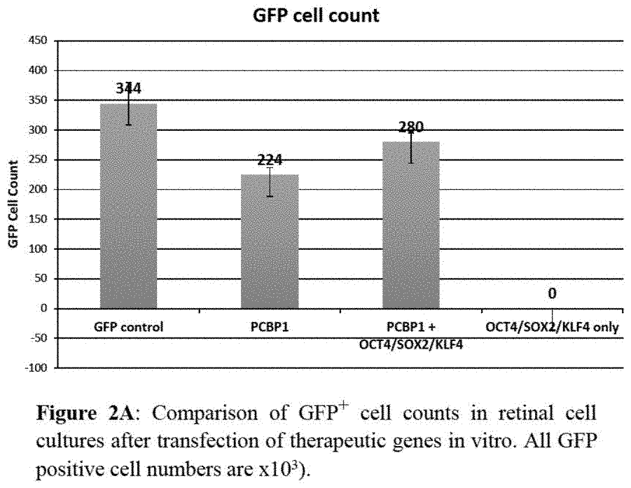

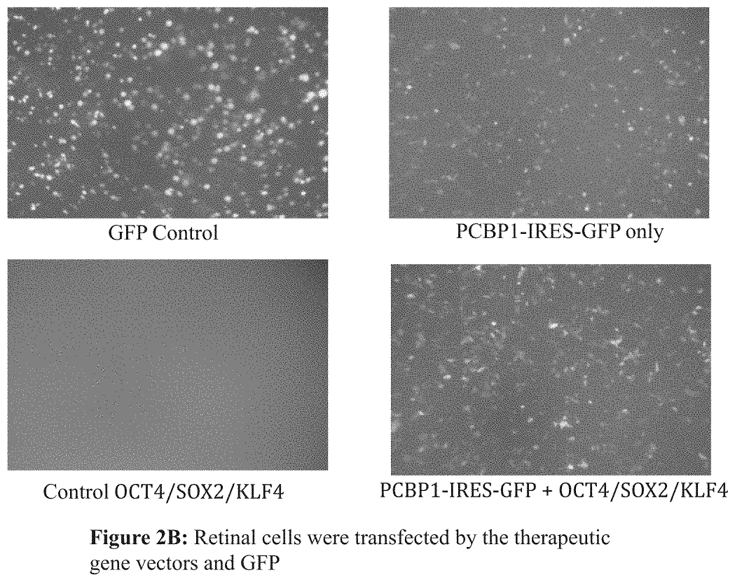

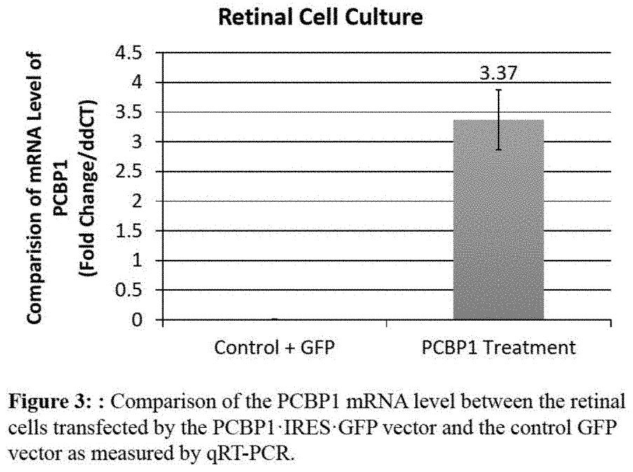

Expression of PCBP1 gene and transcription factor genes into retinal cells: The retinal cells were split and divided into three groups: The first group was transfected with a vector containing PCBP1-GFP (green fluorescent protein) for co-expression of PCBP1 and GFP. After 24-28 hours, expression of GFP was observed, indicating that the vector system succeeded in delivery of all the genes (transcription factors) into the retinal cells (FIG. 2A & 2B); the second group was used to confirm the expression level of the PCBP1 in retinal cells by extraction of total RNA which was subjected to real-time RT-PCR reaction (FIG. 3); and the third group was used for iPSC induction from the differentiated retinal cell, demonstrated as follows. In FIG. 2A, the numbers show more than 90% GFP positive cells. This figure shows a comparison study from the same selected areas (top left (TL), bottom left (BL), top right (TR), bottom right (BR), and center (C)) as FIG. 2B under the fluorescent microscope. In each well, five individual areas were selected and captured by the camera of a fluorescent microscope (top left (TL), bottom left (BL), top right (TR), bottom right (BR), and center (C)). Fluorescent microscopy was then used to observe/capture a photo in each of the 5 areas. The GFP positive cells were counted in each area, and the average calculated in the 5 areas. This average of the 5 areas was multiplied by 10³ (because the area from microscopy capture is around 0.1% of the whole cell area). Therefore, the formula for this method is:

$$Total GFP cell count per well = \frac{( TL + BL + TR + BR + C )}{5} * 1000$$

Twenty eight to seventy-two hours after retinal cell transfection by gene vector, the cells were harvested and their RNA extractions were performed by a Qiagen extraction kit. The isolated total RNA was stored at −80° C. for future use and a cDNA library was generated for RT-PCR using standard laboratory techniques.

Real-time PCR confirmed the expression level of PCBP1 in retinal cells in vitro. As shown in FIG. 3, PCBP1 was successfully and highly expressed in the target cells by the vector encoding the PCBP1.

Stem Cell Culture: Retinal cells were cultured on cell culture plate. PCBP1 (with GFP) and other transcription factors (SOX2/OCT4/KLF4) were transfected into cells by lipofectamine. Transfected cells were cultured for 2-3 days, seeded on Matrigel, and cultured with stem cell growth media (StemRD Catalog No. SPGro-free).

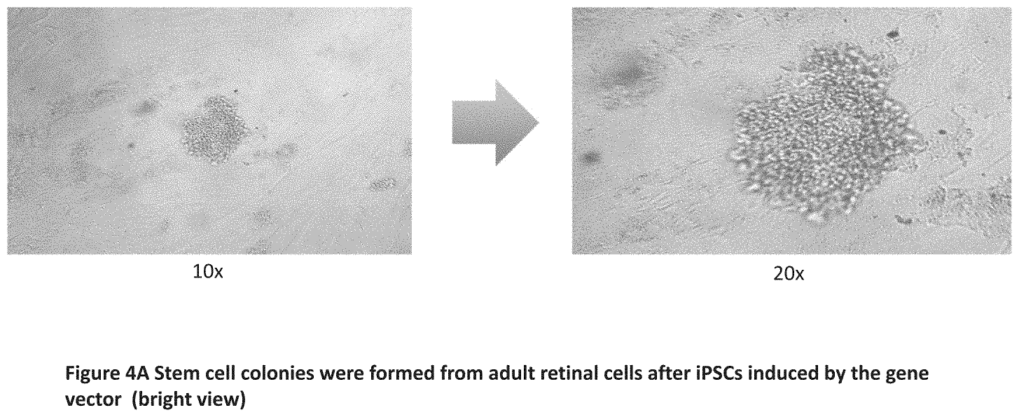

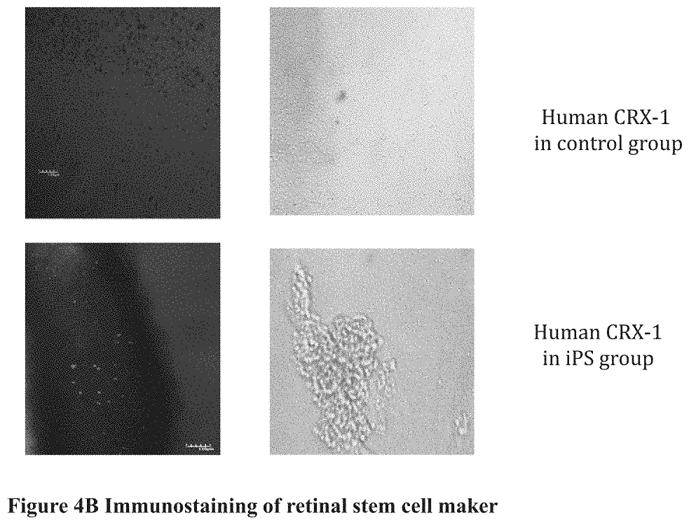

Stem Cell Induced by Transcription Factors (PCBP1/OCT4/KLF4/SOX2): Three commercialized retroviral transcription factors (OCT4/KLF4/SOX2) were used. Two days before viral transduction, 20 μg of C/EBP alpha protein was added on retinal culture cells at 37° C., 5% CO2 incubator. On the day of viral transduction, a suitable amount (final concentration is 10 μM) of beta-estradiol was added on the cell to activate C/EBP alpha-estrogen receptor, and the cells were infected by retrovirus with transcription factors (OCT4/KLF4/SOX2). The cells were incubated for 2-3 days, then the supernatant was collected and the culture was washed twice with 500 μl PBS. The supernatant/PBS was then centrifuged at 300 g for 5 min, the pellet was resuspended in fresh reprogramming medium (human iPS cell growth medium) and plated on a new 24-well plate (well-prepared with matrigel). The cells were incubated in 37° C. incubator for 4 days and stem cell medium was replaced on a daily basis. Daily observation of the cells started at day 4. At day 10-12, colonies should have grown to an appropriate size for analysis, isolation and reseeding. (FIG. 4A).

Antibody Staining and Reorganization: The biomarker CRX-1 may be used as a marker for retinal stem cell reorganization/differentiation. FIG. 4B shows that iPSC cells induced in this experiment express CRX-1. Human anti-CRX antibody (AF7085, R&D system) was applied to cultured cells and conjugated secondary antibody (NL010, R&D system) was added on human anti-CRX antibody as staining signal. Successful binding of antibody resulted in a red fluorescent signal.

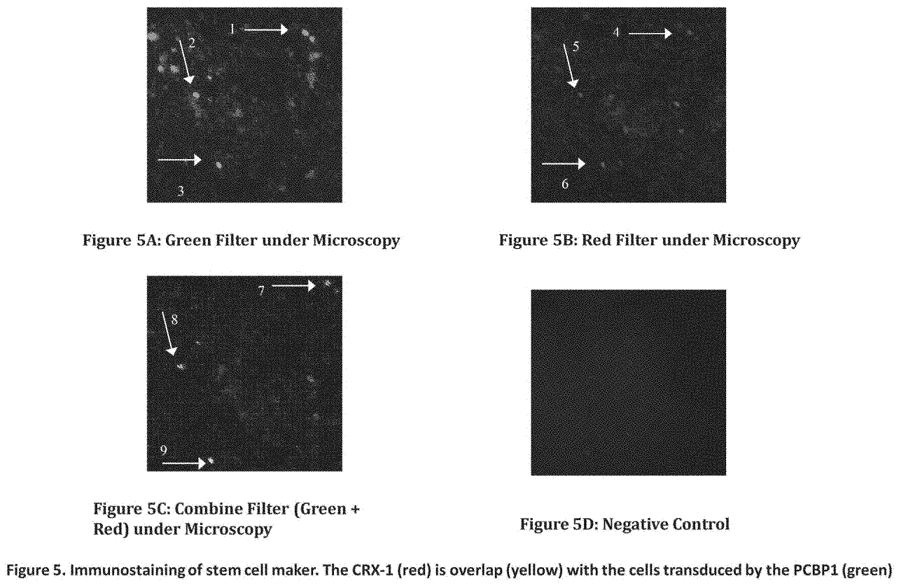

The cells on slides were washed twice by PBS (0.145M NaCl, 0.0027M KCl, 0.0081M Na2HPO4, 0.0015M KH2PO4, pH7.4), then fixed by fixative buffer (4% paraformaldehyde in PBS). After fixation, the slides containing the fixed cells were washed two times in 400 μL of wash buffer (0.1% BSA (bovine serum albumin) in PBS), followed by incubated in permeabilization buffer (0.5% Triton X-100 in PBS) for 5 minutes in room temperature and afterwards non-specific staining was blocked by adding 400 μL of blocking buffer (5% BSA in PBS). The slides were incubated for 45 minutes at room temperature and the blocking buffer was removed. The unconjugated primary antibody (or fluorescence-conjugated primary) was diluted in dilution buffer (1% BSA, 0.3% Triton X-100, and 0.01% sodium azide in PBS) according to the manufacturer's instructions. The secondary antibody was similarly diluted and added to illustrate the primary antibody bound to the target cells. The slides were visualized using a fluorescence microscope and filter sets appropriate for the label used. Slides were stored in a slide box at <−20° C. for later examination. As shown in FIG. 4B, some of the cells turned into retinal stem cell-like cells after transduction with a vector containing PCBP1 and the SOX2, OCT4, KLF4 transcription factors. The antibody staining with one of the retinal stem cell markers shown in FIG. 5 demonstrates that some of the cells have been turned into the retinal stem cell-like cells after transfection with a vector containing PCBP1 and the SOX2, OCT4, KLF4 transcription factors (control group: transfection without PCBP, SOX2, OCT4, KLF4).

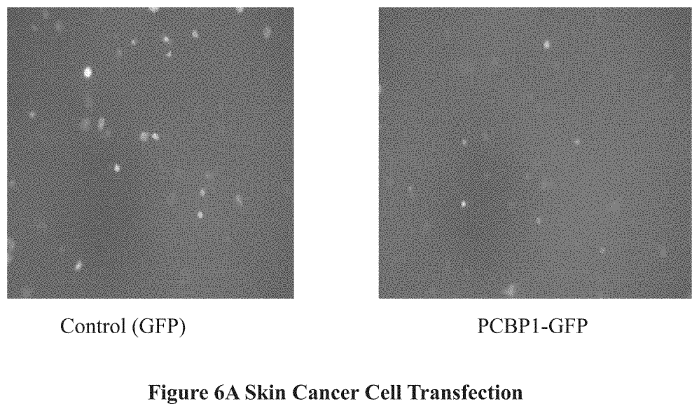

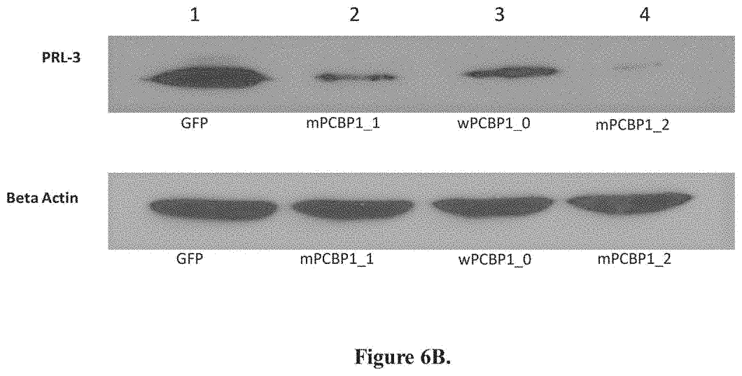

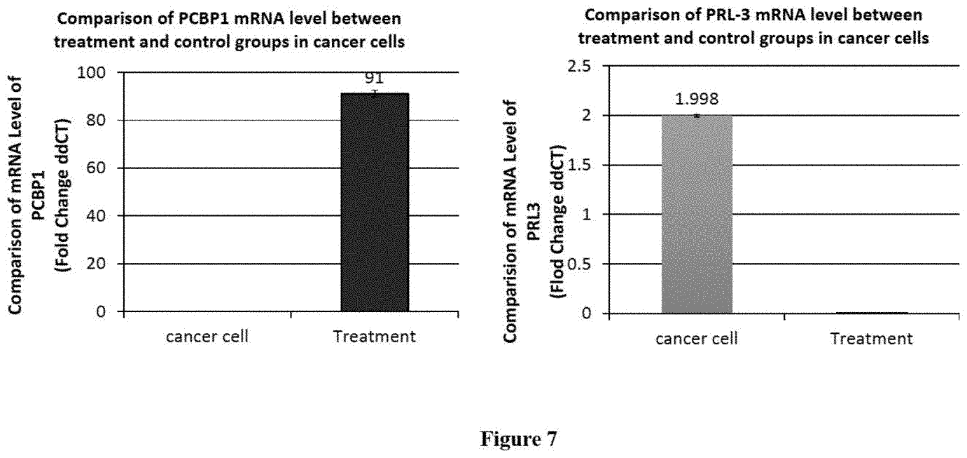

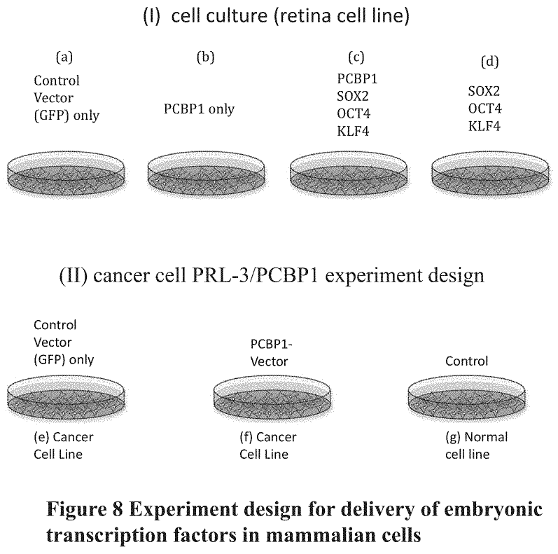

Moreover, the presence of PCBP1 on the vector prevents cancer biomarker expression. FIG. 6 shows the gene vector product is capable to transduce skin cancer cells in vitro. FIG. 7 shows the expression of both PCBP1 and PRL-3 in pancreatic cancer cells. Transduction of the pancreatic cancer cells with PCBP1 inhibits expression of the metastatic marker PRL-3. When compared with the control group, the PRL-3 was down-regulated by overexpression of the PCBP1 mRNA. This was also confirmed at the protein level by Western blot (FIG. 6B).

### Example 2—Injection of Therapeutic Vectors into the Eyes of the Animal Models and Study on Therapeutic Efficacy In Vivo

Transplanted RPE cells show limited adhesion and survival in human eyes, and aged Bruch's membrane are not likely to support adhesion, survival, differentiation, and function of grafted RPE cells. Therefore, the use of genetic engineering to overexpress integrins or integrin activators in the RPE cells or the use of RPE cells growing on scaffolds might show promising prospects. Further, although the subretinal space was once considered to have immune privilege, studies also have indicated that the long-term survival of the transplanted stem cells in the host eyes still require immune suppression. The course of immunosuppression and the drugs used for immunosuppression is being considered in the future clinical applications.

Therefore, the in situ use of iPSC technology to promote retinal cell renewal and regeneration to repair damage will be a practical and improved strategy to treat patients with macular degenerative diseases with minimal or none of the side effects mentioned above. The efficacy of the systems disclosed herein for treating retinal disease will be tested in an animal model. A gene-trap vector will be used to study stem cells in vivo. For example, both AAV and lentiviral viruses may be used to deliver the plasmids of the disclosure. The vectors may be transfected with the plasmids of the disclosure and then purified using standard laboratory techniques.

Animal models: C57B1/6 and DBA/2J mouse will be used in this investigation. For C57B1/6 and DBA/2J mouse, animals will be placed into three major dose groups that include control (GFP only), vector with PCBP1 only, and the PCBP1 vector combined with the three transcription factors SOX2, OCT4 and KLF4 (see Table 3).

All animals will be maintained in the animal facility according to the NIH guidelines under a 12-hour light/dark cycle.

TABLE 3
Animal Model Experiment Design

| | | Animal numbers and drug | | | |
|---|---|---|---|---|---|
| | | delivery | | | |
| | | Control | PCBP1, +SOX2, | PCBP1 | |
| Animal | | GFP gene | Oct4, and | gene- | |
| models | Species | only | KLF4 genes | GFP | Note |
| Mouse | C57Bl/6 | 5 | 5 | 5 | Triplicate of |
| | | | | | total animal |
| | | | | | number will |
| | | | | | be 45 |
| Mouse | DBA/2J | 5 | 5 | 5 | Triplicate of |
| | | | | | total animal |
| | | | | | number will |
| | | | | | be 45 |

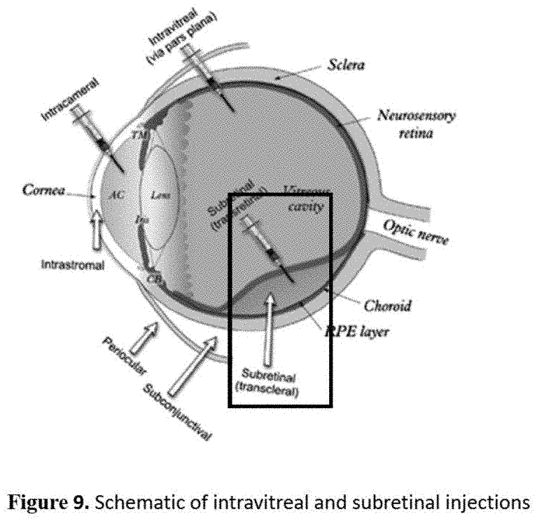

In vivo drug delivery into eyes of animals: Administration of the vector constructs into the retinal layer of the eyes in the rat and mouse models will be by subretinal injection (FIG. 9).

Spatial Visual Acuity: To test whether repair of damaged retinal cells by delivery of the gene vector can restore the vision from the macular generation, the animals will be tested for visual acuity using an optometry testing apparatus (Cerebral Mechanics, Lethbridge, Canada). This test comprises four computer monitors arranged in a square, which project a virtual three-dimensional space of a rotating cylinder lined with a vertical sine wave grating. Unrestrained animals are placed on a platform in the center of the square, where they track the grating with reflexive head movements. The spatial frequency of the grating is clamped at the viewing position by recentering the “cylinder” on the animal's head. The acuity threshold is quantified by increasing the spatial frequency of the grating using a psychophysics staircase progression until the following response is lost, thus defining the acuity. Rats are tested at monthly intervals. Elov14 mice were also tested in this apparatus at 3, 5, 7, and 11 weeks after injections (see FIG. 10). The version result can be adjusted/analyzed by cycle/degree. Higher cycle/degree indicates better version recovery (i.e., normal rat version is 0.6 cycle/degree).

Assessment of cancer development in treated animals: The long-term risk of teratoma formation is tested in the NIH III mouse model, chosen for its immune-deficient status. The nude mouse has three mutations rendering it devoid of T cells, NK cells, and mature T-independent B lymphocytes. However, the NIH III mouse retains eye pigmentation, which provides better visualization for subretinal injection. The surgical technique is the same as performed in the RCS study. The study compares the treatment to control groups over three time points: 1, 3, and 9 months (the approximate lifespan of the animal; n=6 per cohort. No teratoma or tumor formation is found in any of the animals injected with the viral gene vector in the safety study by the following studies. Teratoma or tumor formation may be assessed via histochemistry or drug distribution studies as discussed below.

Histochemistry (cancer or tumor, tissue structure): At the end of functional studies, all animals are killed with an overdose of sodium pentobarbital and perfused with phosphate-buffered saline. The eyes are removed, immersed in 2% paraformaldehyde for 1 hour, infiltrated with sucrose, embedded in optical cutting temperature, and cut into 15 μm horizontal sections on a cryostat. Four sections (50 μm apart) are collected per slide, providing five series of every fourth section collected. One is stained with cresyl violet for assessing the injection site and integrity of retinal lamination. The remaining slides are used for antibody staining, for cancer/tumor detection, and tissue structure analysis.

Drug distribution and analysis of the pathways of drug actions in retinal tissues: Global gene expression analysis is performed using the human and mouse or rat Affymetrix HG-U133 Plus 2.0 microarray platform (Santa Clara, Calif.) on PCBP1 expression from different organs/tissues that involve delivery of the drug. Additionally, ARPE-19 (American Type Culture Collection™, Manassas, Va.), and retinoblastoma (American Type Culture Collection™) cell lines and normal retinal cells are analyzed as controls when compared with treatment. Data analysis will be supported by statistical software.

Immunoblot analysis of PCBP1 and other transcription factors is carried out using standard SDS-PAGE methods using the Bio-Rad Mini-Protean and Mini-Transblot Cell (Hercules, Calif.). The protein bands are visualized using Western Lightning Chemiluminescence Reagent (PerkinElmer Life and Analytical Sciences, Boston) and a Kodak 4000MM digital imaging station (Rochester, N.Y.).

### Example 3—PCBP1 Interacts with Sox2, Oct4, KLF4 and Others Via PRL3/STAT3 Pathways

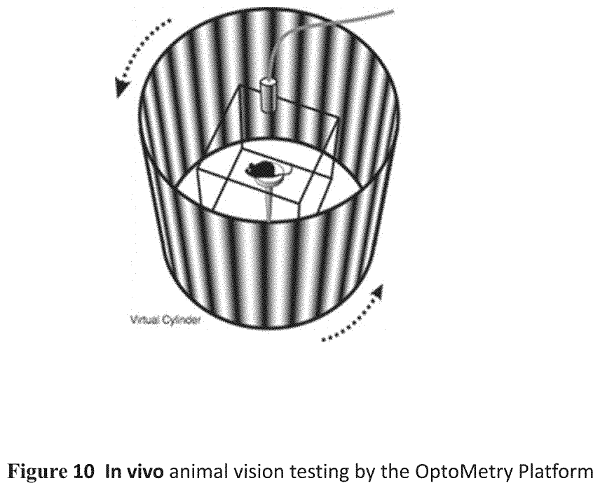

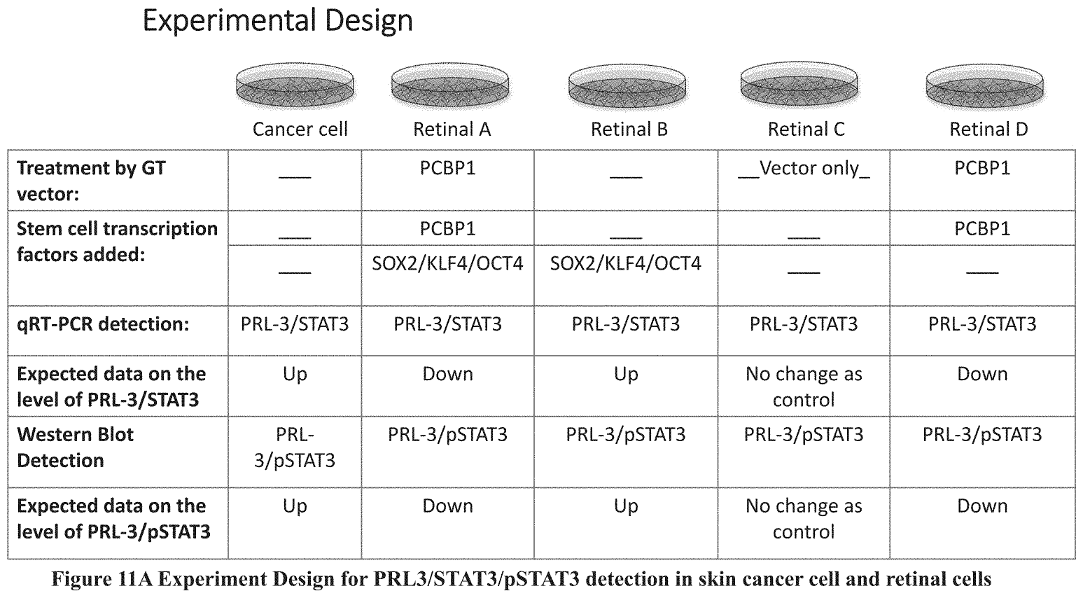

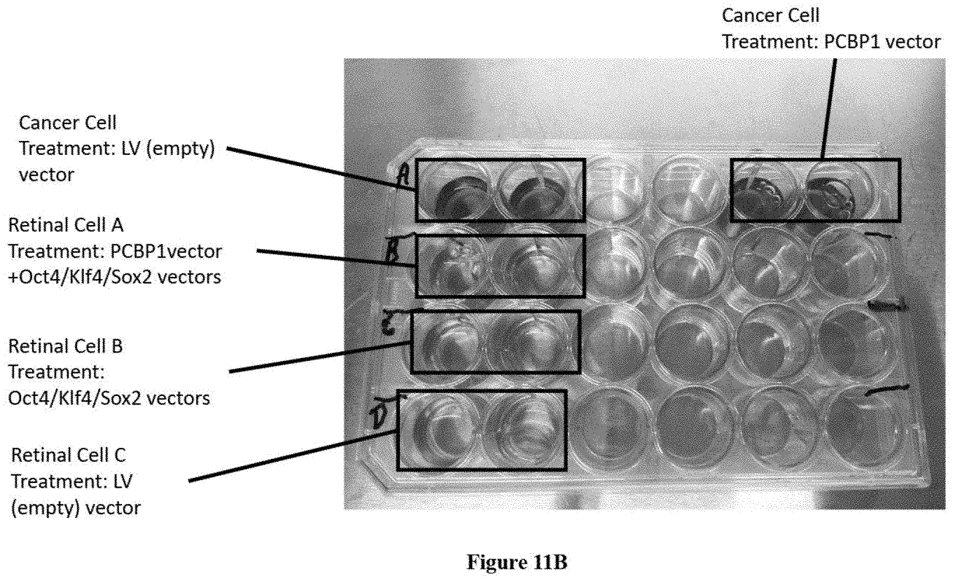

To study the interaction of PCBP1 with other stem cell transcription factors and determine the pathway by which they interact, studies in retinal cells may be performed as follows. Four cell cultures are treated with various vectors of the present disclosure. As shown in FIG. 11A, a culture of cancer cells is used as a control and not transfected with any vector. qRT-PCR of this culture will show expression of biomarkers associated with cancer and metastasis (PRL-3/STAT3). Three cultures containing retinal cells will be transfected as shown in FIG. 11A. In the cell culture transfected with vectors containing PCBP1 and the SOX2/KLF4/OCT4 stem cell transcription factors, the expression of the PRL-3/STAT3 is expected to be inhibited compared with the expression levels in the cancer cells or nontransfected retinal cells. In the cell culture transfected with vectors containing the SOX2/KLF4/OCT4 stem cell transcription factors, the expression of the PRL-3/STAT3 is expected to be increased compared with nontransfected retinal cells and those additionally transfected with PCBP1. FIG. 11B shows a representative plate used in this experiment.

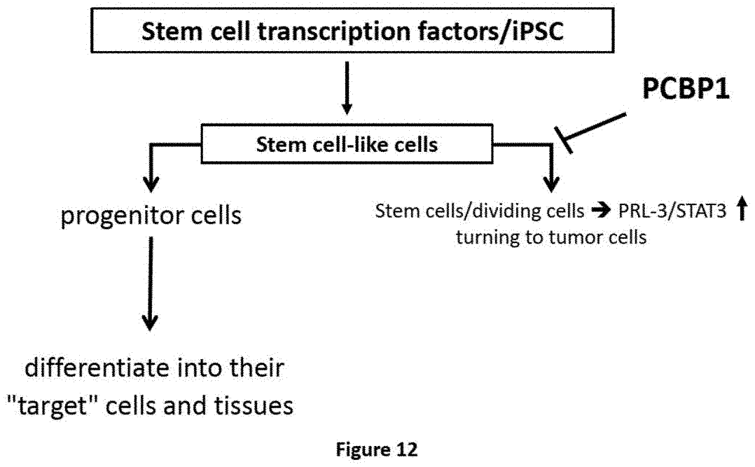

FIG. 12 shows a putative mechanism by which the stem cell transcription factors and the PRL-3/STAT3 pathway interact. During iPSC generation, PCBP1 works with stem cell transcription factors (e.g. SOX2, OCT4, KLF4) to maintain stem cell development. However, PCBP1 also acts on pathways inhibiting PRL-3 linked to down-regulation of the STAT3 signal to promote progenitor cell differentiation into target cells or tissues, without increasing tumorigenesis.

### Example 4—Cardiac Fibroblast Stem Cell Induction In Vitro

There are more than 1.5 million cases of myocardial infarction in the United States each year, with the irreversible death of cardiomyocytes (CMCs) secondary to insufficient oxygen supply causing great injury. CMCs in the heart have limited regenerative abilities, and as a result, about five million patients who survive acute myocardial infarction suffer from ischemic cardiomyopathy, resulting in heart failure. Therefore, an effective therapy for acute myocardial infarction to repair the scarred tissue by replenishing the damaged CMCs is required.

Direct fibroblast reprogramming was first attempted via viral overexpression of cardiomyogenic factors such as GATA4, Mef2C, and Tbx5 (GMT) (Chen J X et al. (2012)). Recently, Ma et al. (2017) further optimized the strategy by varying protein stoichiometry to achieve higher efficiency in the generation of mature CMCs. Subsequent modification of the therapeutic strategy increased the reprogramming efficiency up to 60% through small molecule inhibition of pro-fibrotic signaling. However, alterations in GATA4 gene expression have been associated with several cancer types, thus increasing tumorigenesis potential.

Stem cells that are induced in situ to specifically target the damaged cells facilitate tissue repair. Induction in situ can avoid the common problems associated with ex situ stem cell induction before transplantation, such as target area inaccuracy and pathogen contamination. Shown herein is direct conversion of cardiac fibroblasts (CFs) into CMCs that significantly lowers the risk of side effects, including tumorigenesis.

### In Vitro Induction of Cardiac Fibroblast Cells into Stem Cell-Like Cells

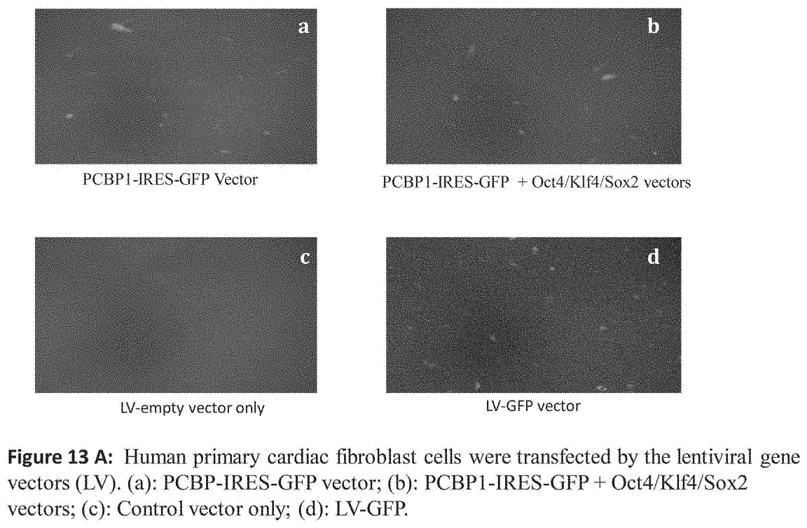

Individual lentiviral vectors were constructed expressing co-expressing PCBP1 and GFP downstream of a target gene (PCBP1-IRES-GFP) and separately expressing Oct4, Klf4, Sox2, and c-Myc. Human fetal cardiac fibroblast cells (FCF) were grown in medium without FBS and then split for lentiviral transduction. Subsequently, they were divided into three groups. Group 1 was transduced by LVV PCBP1-IRES-GFP with or without Sox2/Oct4/Klf4 for GFP visualization, or with a GFP-only vector used as a positive control (FIG. 13A). Group 2 was used to confirm the expression of the PCBP1 mRNA in cardiac fibroblast cells by total RNA extraction and qRT-PCR analysis (FIG. 13B). FIG. 13B shows a comparison of the PCBP1 mRNA levels of CFs transduced by the PCBP1-IRES-GFP vector and control (no transfection) by qRT-PCR. The transfected cells show more than a 99-fold higher PCBP1 expression (ddCt=99.9; p<0.05).

The mRNA of the other transcription factors was also confirmed by qRT-PCR (data not shown). Group 3 was used for iPSC induction of the FCFs into cardiac stem cell-like cells.

The FCFs from Group 3 were continuously grown and then transduced by lentiviral vectors for 72 hours before being transferred onto new plates containing matrigel as a substrate, and grown in stem cell medium without FBS in the presence of c/EBP-Alpha and beta estradiol for 16 days.

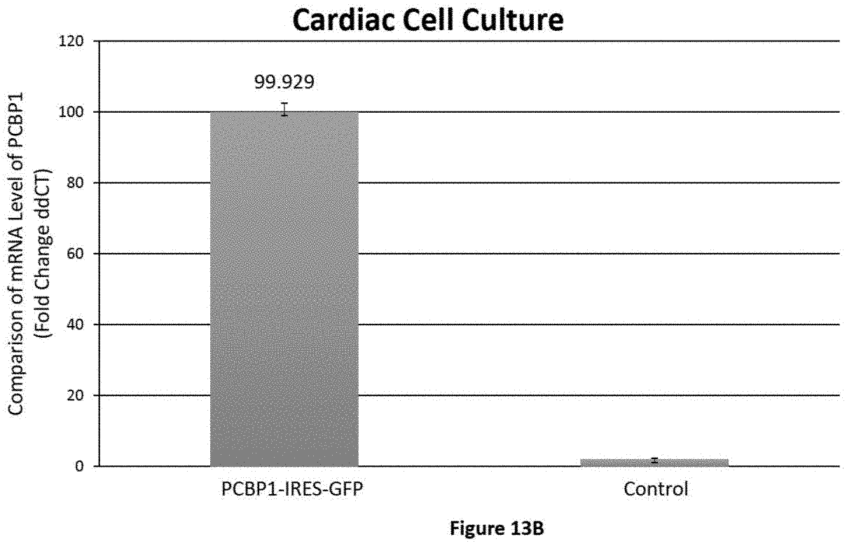

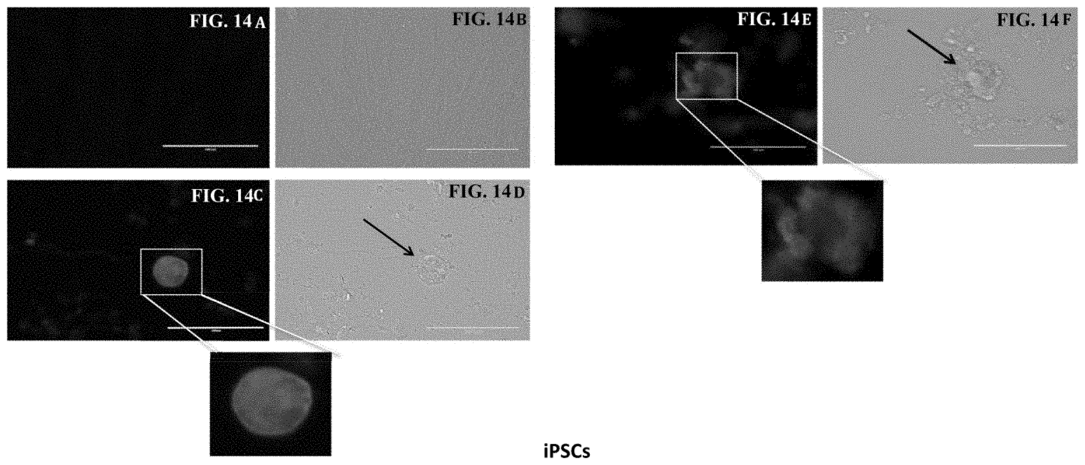

The c-Kit receptor tyrosine kinase has been shown to enable heart cells in culture to differentiate into CMs, smooth muscle, and endothelial cells, and is used as a cardiac stem cell marker. An antibody specific for c-Kit was used to detect the emerging cardiac stem cell biomarker and identify cardiac stem cell-like cells as shown in FIG. 14. Sixteen days post-transduction, the FCFs lost their typical spindle structure and displayed as phenotypic cardiac stem cell sphere-like structures. These FCFs were also positively detected by the c-Kit antibodies (FIG. 14). Interestingly, transduction of CFs with LVV expressing PCBP1 alone effectively induced the cardiac fibroblasts to change phenotype from the classic spindle-like structure to a structure similar to that described for cardiospheres, a source of cardiac stem cells and proven to have regeneration capacity (FIG. 14C and D).

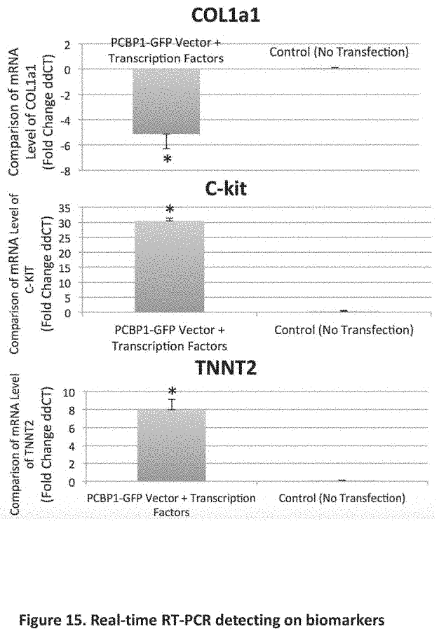

As a CF becomes stem cell-like, it gradually loses the biomarkers specific to fibroblasts and expresses cardiac stem cell biomarkers on the cell surface. For example, COL1a1 is a cardiac fibroblast specific marker gene. TNNT2, which encodes the cardiac muscle troponin T, is a biomarker for CMC and is enriched in cardiac crescent-stage progenitor cells. As shown in FIG. 15, transduction with LVVs containing PCBP1, Sox2, Oct4, and Klf4 resulted in more than a 5-fold reduction of COL1a1 mRNA levels. Moreover, transduction with the LVVs induced a more than 30-fold increase in c-Kit expression and an almost 8-fold increase in TNN2 expression.

These results demonstrate that CFs can be induced by LVVs containing PCBP1, Sox2, Oct4, and Klf4 in vitro to generate cardiac stem cell-like cells.

### Example 5—Cardiac Fibroblast Stem Cell Induction In Situ

Differentiated fibroblast cells can be reprogrammed to induce pluripotency and become stem cells. Current stem cell therapies require ex vivo modifications prior to infusion or implantation, primarily due to the need to deliver molecules that reprogram gene expression patterns and induce pluripotent stem cells (iPSCs). While this is typically done in the laboratory on isolated cells, it is difficult to maintain the stem cell state during ex vivo manipulations. Here, instead of inducing iPSCs ex vivo, induction will occur in situ, e.g. directly in the heart using co-expression of PCPB1 and one or more transcription factors. The combined expression of PCBP1 and the iPSC transcription factors will directly induce conversion of the cardiac fibroblasts into stem cell-like cells and eventually CMC for the treatment of myocardial infarction.

Using molecular cloning techniques, the four transcription factors involved in iPSCs were engineered to be expressed by a single vector system, facilitating delivery of the transcription factors and enabling reproducible results. The expression of each individual gene will be tested by qRT-PCR for mRNA and by Western blot for protein analyses.

Mouse heart tissue will be surgically treated to produce a myocardial infarction. The vector systems of the disclosure will then be transduced in differentiated fibroblasts in situ to trigger the transformation of the CFs into stem cells, which will be detected by stem-cell specific markers. Several in vitro functional assays will be conducted as follows:

Immunocytochemistry: iPSCs will be induced in cultured CFs, visualized by bright filed and confirmed by detection of cardiac stem cell biomarkers. The FCFs will be transduced by the LVV vector system expressing pooled individual virus and polycistronic PCBP1, Sox2, Oct4, and Klf4 viruses in vitro. Additionally, a LVV vector containing only PCBP1 (i.e. with no other transcription factors) will be tested because of the cardiosphere structure observed from the study in Example 4. Four days post-transduction, the cells will be transferred onto a matrigel coated dish and cultured for 15 days to induce cardiac stem cells. After the cardiac stem cell clusters are visible (compared with a control group, immunohistochemistry with an antibody specific for c-Kit will be performed to detect c-Kit expression levels under fluorescence microscopy.

Evaluation of cardiac reprogramming in vitro—CMC beating assay: iPSCs will be induced as described above. Fluorescence imaging of intracellular calcium levels will be analyzed by automated signaling analysis software. The CMC are expected to demonstrate the phenotypic response of spontaneously contracting cells, with analysis of the parameters indicative of cardiac physiology.

Evaluation of tumorigenesis potential in vitro—Signaling pathway analysis: Phosphatase of Regenerating Liver 3 (PRL3) is an oncogenic factor, the activation of which is often associated with tumorigenesis. Likewise, Stat3 is involved in carcinogenesis of a variety of cancers. Evaluation of these oncoproteins is important in mapping out the signaling pathway regulated by the current LVV system. The total RNA and proteins will be extracted and then subjected to analysis for specific biomarkers such as c-Kit, COL1a1, TNNT2, PRL3, and Stat3 using qRT-PCR and Western blot for total proteins and phosphorylated/activated forms. Down regulation of PRL3 total and activated protein levels and decreased activation of downstream target STAT3 indicate reduced oncogenesis potential.

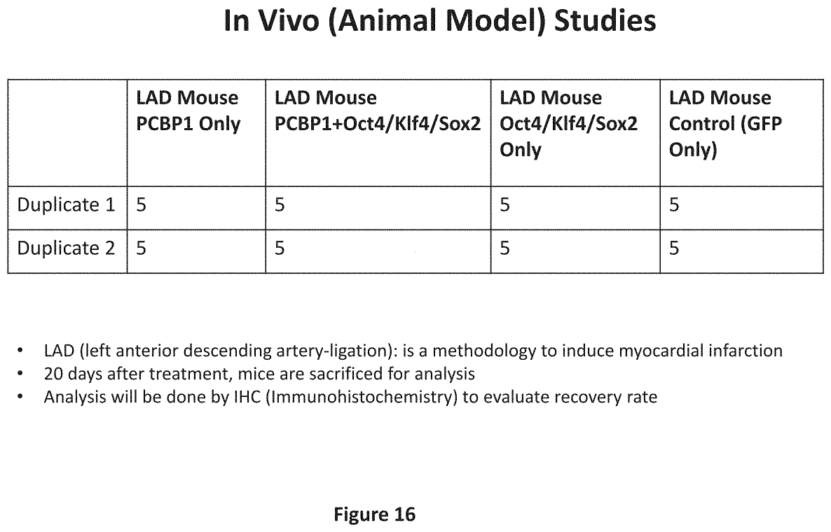

Efficacy in myocardial infarction murine model: Animal testing will be conducted to confirm the applicability of the system in vivo (see FIG. 16). Lentiviral vectors will be injected into the heart tissue of an MI animal model to induce CMC regeneration. Successful heart function recovery will be monitored by behavioral study, and physiologic and histological analysis. Assessments of signaling pathway components at the molecular level will also be performed.

In vivo models: the left anterior descending (LAD) artery-ligation murine model for MI is used to reproduce the local ischemic myocardial tissue damage that is typical in human heart attacks. Previous studies using a mouse MI model delivered Gata4, Mcf2c, and Tbx5 in a single mRNA via a retroviral vector. Here, a lentiviral vector is used which allows for delivery of a larger gene payload. There will be four cohorts for the animal study using LAD-ligation in Balb/C mice: 1) PCBP1; 2) PCBP1/Oct4/Klf4/Sox2; 3) c-Myc/Oct4/Klf4/S0x2; and 4) control vector expressing GFP as a negative control. Five animals will be included in each cohort, and experiments will be repeated three times. Twenty days after the injection of virus, the behavior of the animals will be recorded for recovery from heart attack. The animals will then be anesthetized and sacrificed. The explanted hearts will be analyzed by immunohistochemistry for recovery in the ischemic area. Global microarray gene expression profiling will be monitored for signaling pathway components downstream of the PCBP1 and Sox2, Oct4, Klf4 transcription factors.

Evaluation of direct reprogramming in vivo—Determination of scar size: Heart tissue will be stained with standard Masson-Trichrome staining 8 weeks after MI. The infarcted area shows in blue, while viable myocardium is red. The scar area on a serial of transverse sections with the first level right below the ligation will be analyzed using Image J software.

Evaluation of direct reprogramming in vivo—Cardiac function: The echocardiography, hemodynamics, and MRI of the hearts of experimental animals will be analyzed to evaluate the cardiac function at 4, 8, and 12 weeks after MI with or without LVV injection.

Evaluation of direct reprogramming in vivo—Gene regulation: CMC will be isolated from different heart regions and time points after MI and treated by digestion and FACS. Total RNA and protein will be harvested and analyzed for signaling pathway components such as Stat3 and/or PRL3. The group injected with LVV containing PCBP1, Sox2, Oct4, and Klf4 will show comparable or even better recovery of heart functions compared with the c-Myc, Sox2, Oct4, and Klf4 group. Both the LVV transcription factor groups are expected to recover better than the control groups, and there should be no significant difference between the GFP and the saline control group.

Alternative vector construction strategy: Because of the accumulative large size of the four transcription factor proteins, they may need to be delivered individually due to payload capacity limitations. Therefore, the following alternative construction options may be used. (1) Re-engineering the construct, by generating two fusion proteins of “PCBP1-2A-Sox2” and “Oct4-2A-KLF4”, the expression of these two fusion proteins will be driven by two separate EF promoters, and each protein will be equally released within cell after delivery; (2) Constructing four individual vectors expressing each transcription factor, respectively.

If stem cell-like cells in the in vivo model cannot be induced using a single vector, the following alternative plans may be applied: (1) Use different constructs—Pool the virus containing four individual vectors together for delivery into the targeting tissue since that individual vector has been proven to express transcription factors in the previously made constructs; (2) Selection of another delivery system such as Adeno-associated Virus vector (AAV) is another alternative. Four AAV vectors may be constructed, each encoding one transcription factor. The advantage of the AAV vector is that they can deliver much higher virus titers and local protein concentration, which might lead to higher efficacy on the target cells. Furthermore, AAV vector has been approved by FDA for clinical trial on therapeutics for patients with advanced heart failure; (4) Add inhibitors of pro-fibrotic signaling such as TGF beta and ROCK signaling to increase the efficiency.

### Example 6—FACS Detection of CD44 and CD24 in MCF-7 (CSC) Breast Cancer Stem Cells (BCSC) vs. MCF-7 Cancer Cells

TGF-beta culturing: MCF-7 breast cancer cells stem cells and MCF-7 cancer cells were treated using complete medium supplemented with TGF-beta (5 ng/mL) for 7 days after incubation to enhance cancer stem cell production and purification by selection for CD44+ and CD24− sub-populations. Selected cells were cultured (e.g. with TGF-beta+ medium) until the cell numbers were sufficient for experiments (typically about 4 to 5 days post-isolation). Control cells (i.e. those not administered TGF=beta) were treated as normal with the recommended medium. All transfections were conducted with Lipofectamine 3000. Mock transfections were conducted using the Lipofectamine treatment without vector DNA.

Harvested cells were co-stained with anti-human CD44 PE-conjugated monoclonal antibody (10 μL/106 cells) and anti-human CD24 APC-conjugated monoclonal antibody (10 μL/106 cells). A non-specific antibody was used as an isotype control.

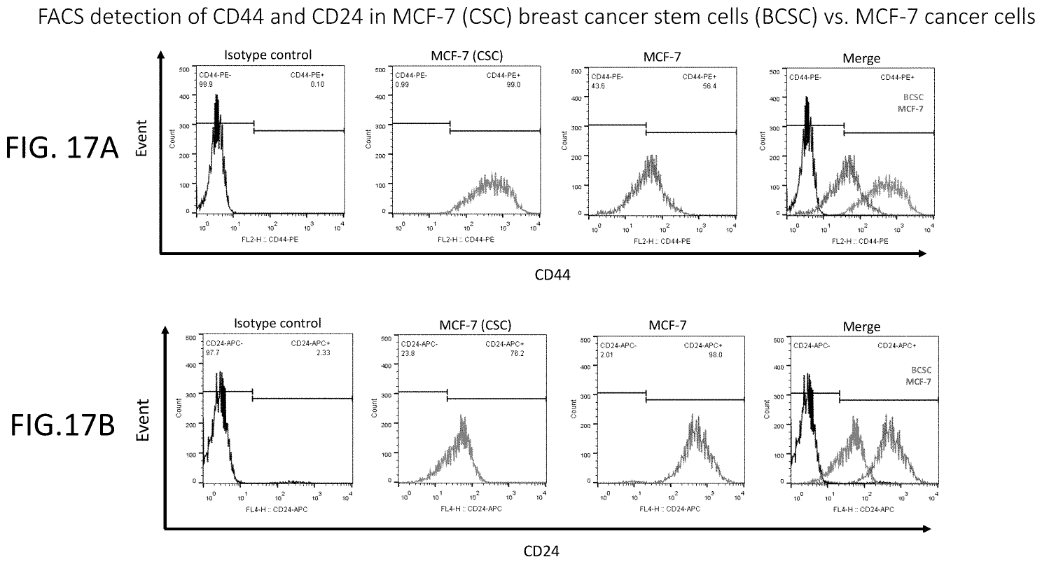

FIG. 17 shows the MCF-7 (CSC) (BCSC) have higher CD44 expression, but decreased CD24 expression, thus validating the enrichment of breast cancer stem cell populations (CD24−; CD44+) using this method.

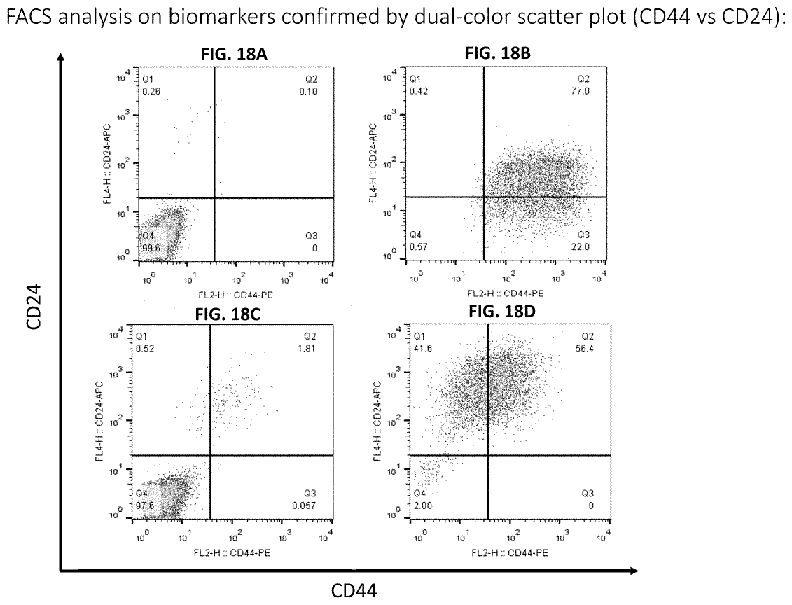

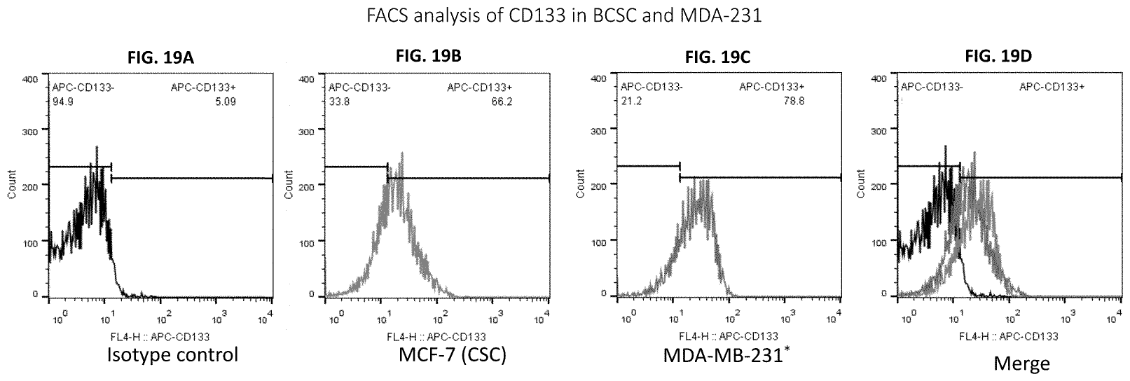

The cells were also analyzed for expression of CD24 and CD44 via dual-color scatter plots. Cells were harvested and co-stained with anti-human CD44 PE-conjugated monoclonal antibody (10 μL/10⁶ cells) and anti-human CD24 APC-conjugated monoclonal antibody (10 μL/10⁶ cells). Non-specific antibody was used as a control. FIG. 18A-D. Further, FIG. 19A-D shows that the MCF7 cell line expresses less CD133 than the MDA-231 breast cancer cell line.

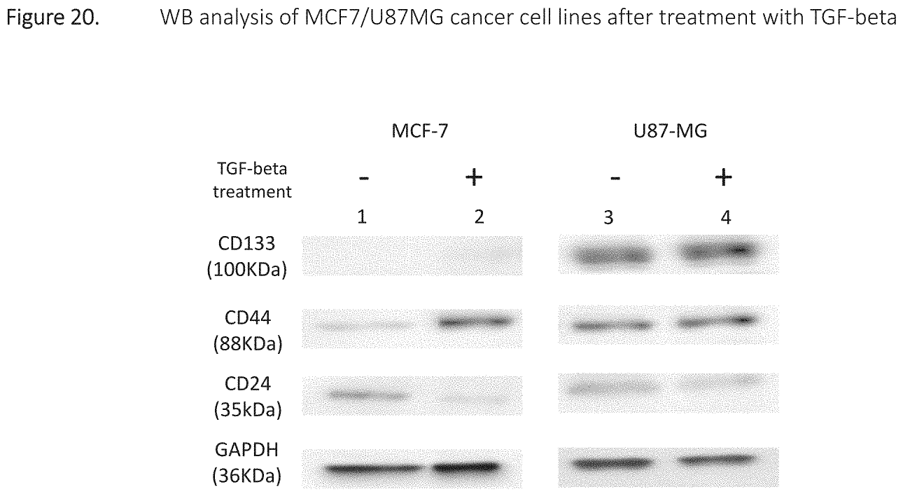

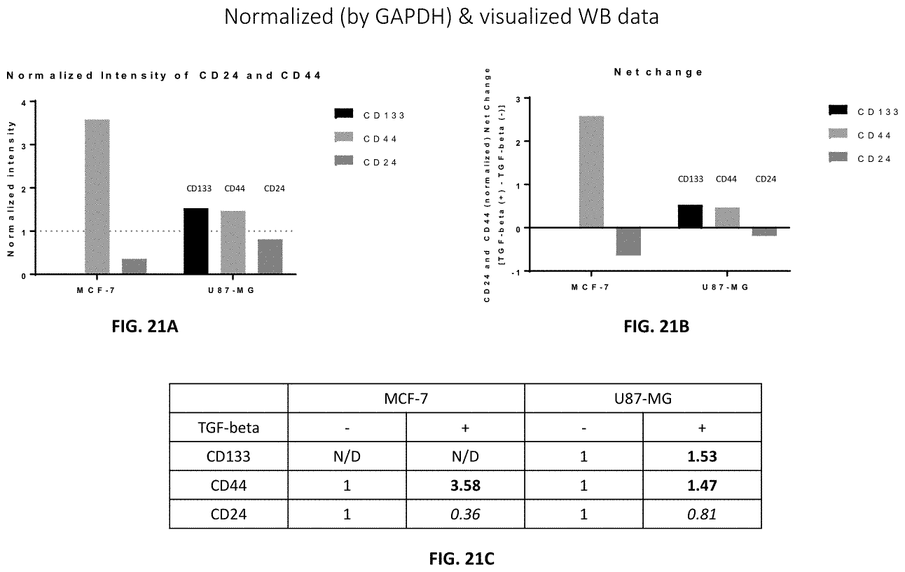

To determine the effect of TGF-beta on the various cell lines, MCF-7 and U87-MG (human glioblastoma) cells were lysed by RIPA lysis buffer and 35 mcg protein (determined by BCA assay) of cell lysate was loaded per well. Rabbit anti-CD133 antibody (1:1000), murine anti-CD44 monoclonal antibody (1:1000), and sheep anti-CD24 antibody (1 μg/mL) were used as primary antibodies. Secondary reagents were goat-anti-rabbit HRP-conjugated antibody (1:1000), goat-anti-mouse HRP-conjugated antibody (1:1000); and donkey-anti-sheep HRP-conjugated antibody (1:1000). For normalization and quantification, after primary probing the blot was stripped and incubated with rabbit anti-GAPDH antibody (as internal control) (1:3000). The second antibody for GAPDH was goat-anti-rabbit HRP-conjugated antibody (1:1000). FIG. 20 shows this western blot analysis of MCF7/U87MG cancer cell lines after treatment with TGF-beta. These Western Blot data were normalized by GAPDH. See FIG. 21A-C.

### Example 7—Effects of PCBP1 Overexpression in Cancer Stem Cells

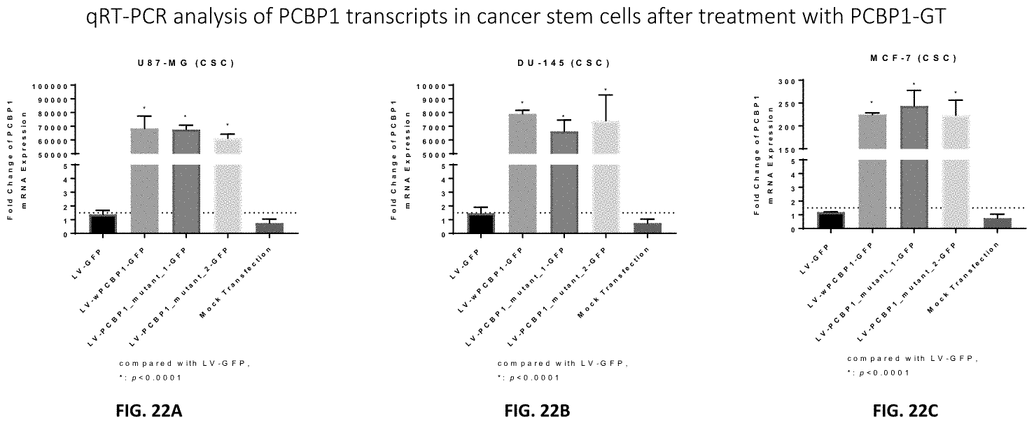

Various cancer cell lines were transfected with PCBP1 constructs, LV-GFP, wPCBP1_0, m_PCBP1_1, mPCBP1_2, or mock-transfected, as described herein. The amount of PCBP1 transcripts in the transfected cells was analyzed by qRT-PCR. FIG. 22. Total RNA was extracted from cells 48 hours post-transfection, followed by conversion to cDNA using reverse-transcription PCR. PCBP1 and variants were measured by qPCR with a PCBP1-specific Taqman probe. GAPDH served as an internal control. There is no significant difference of PCBP1 mRNA expression among LV-wPCBP1-GFP (wild type), LV-PCBP1_mutant_1-GFP and LV-PCBP1_mutant_2-GFP in all three cell lines. Mock-transfection served as base-line for calculation.

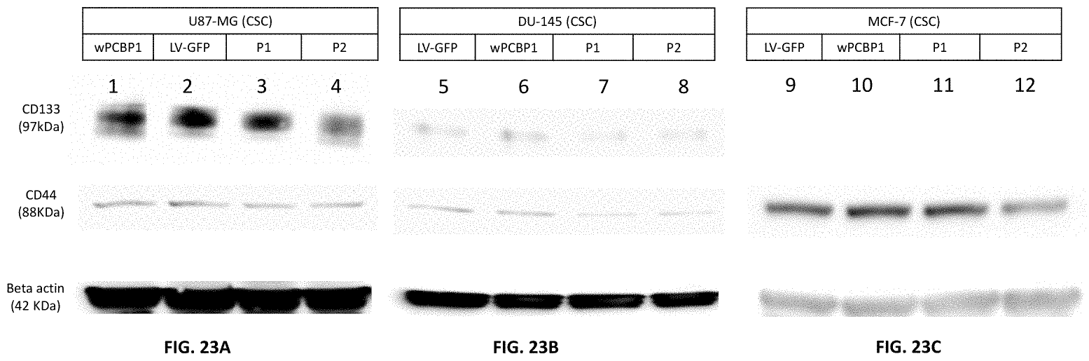

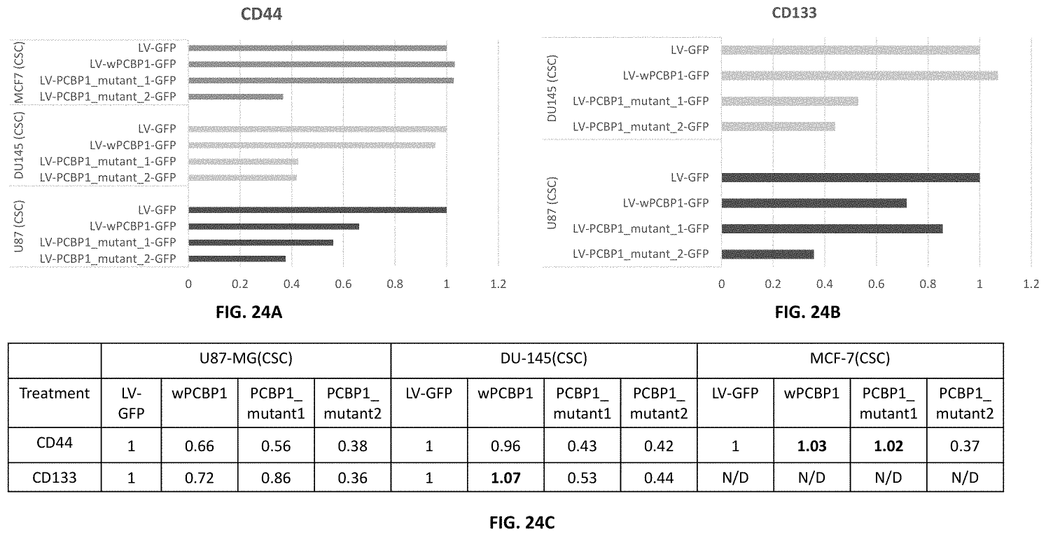

The biomarker expression in the cancer stem cells after transfection with the PBCBP1 constructs of the disclosure is shown in FIG. 23. Cells were lysed with RIPA lysis buffer and 35 mcg protein (determined by BCA assay) of the cell lysate loaded per well. Rabbit anti-CD133 antibody, (1:500) and murine anti-CD44 monoclonal antibody (1:1000) were used as the primary antibodies. Goat-anti-rabbit HRP-conjugated antibody (1:1000) and goat-anti-mouse HRP-conjugated antibody (1:1000) were used as the secondary reagents. For normalization and quantification, after primary probing the blot was stripped and probed with mouse anti-Beta actin monoclonal antibody (0.01 μg/mL). The second antibody for beta actin was goat-anti-mouse HRP-conjugated antibody (1:1000). FIG. 23A-C shows that both CD44 and CD133 are significantly down-regulated after treatment. FIG. 24A-C shows the normalized (against beta-actin) expression of CD44 and CD133 in the transfected cells.

Summary: Cancer stem cells were generated and isolated using TGF-beta treatment and sorting for a CD24− and CD44+ populations (confirmed by FACS and Western blot). After transfection of those isolated cancer stem cells with either wild type or mutant forms of PCBP1, CD44 and CD133 were significantly decreased in cancer stem cells after expression of either the wild type or mutant forms of PCBP1. CD44 can be used as a cancer stem cell biomarker and CD133 can be used as a cancer cell biomarker. However, as demonstrated here, expression of wild type or mutant PCBP1 can block the expression of CD44 and CD133 to prevent the tumorigenesis potential and sternness of the cancer stem cells. This indicates that PCBP1 may play an important role as a regulator during the generation of iPCS.

### Example 8—Niclosamide or PCBP1 Expression Downregulates BCL-2 and c-Myc

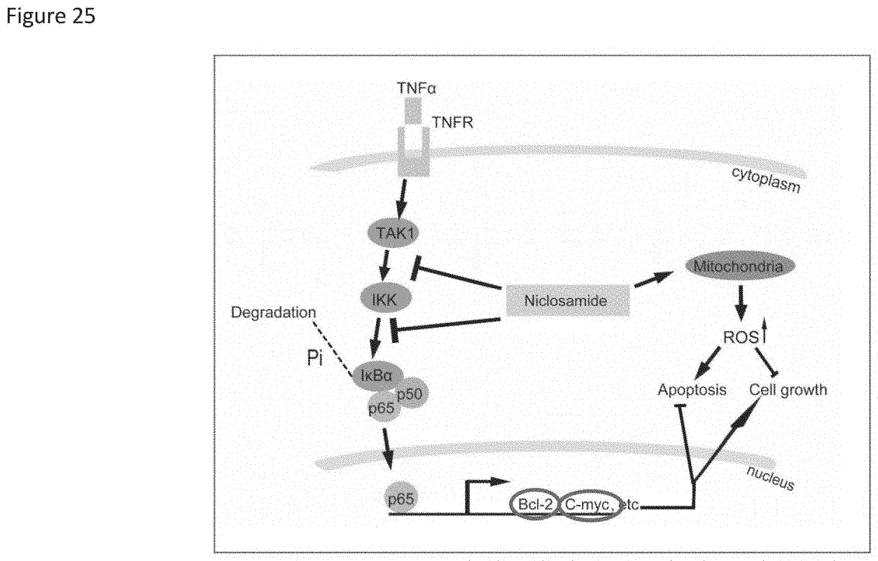

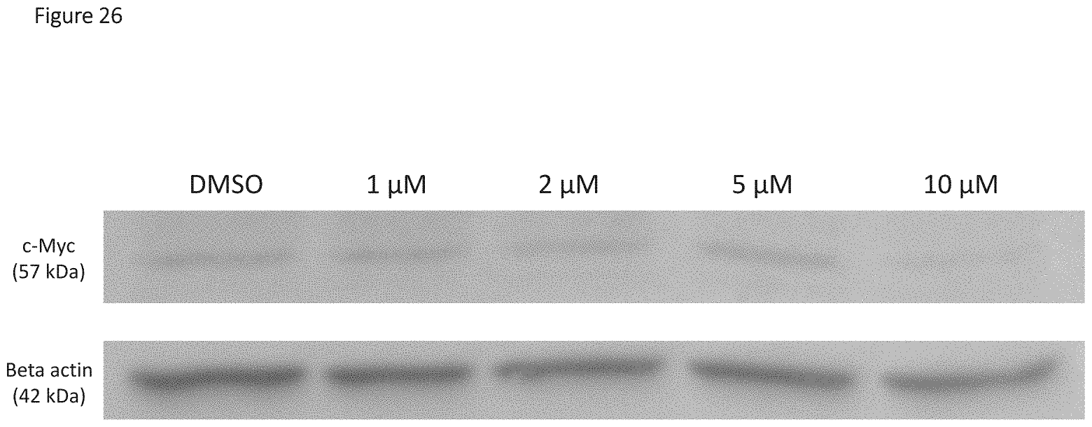

Niclosamide has been found to have inhibitory potential on cancer stem cells, involving various pathways. We therefore studied niclosamide as a cancer stem cell inhibitor. Many relevant pathways were shared between cancer stem cells and stem cells (e.g., maintaining an undifferentiated status, instead of inducement to the generation of PSCs). Here, the effect of niclosamide on the NF-kB pathway was studied. FIG. 25.

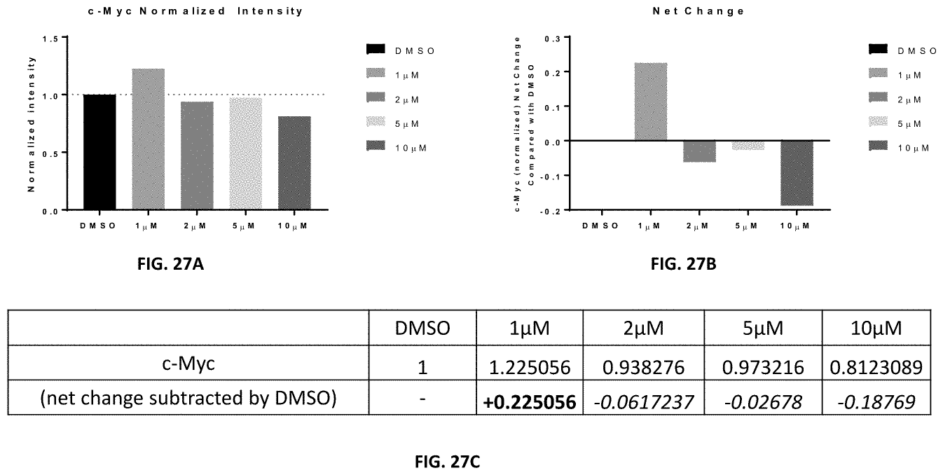

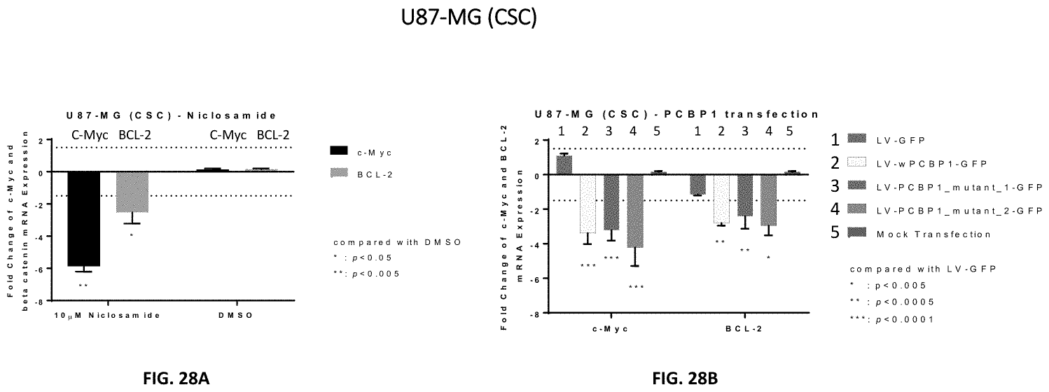

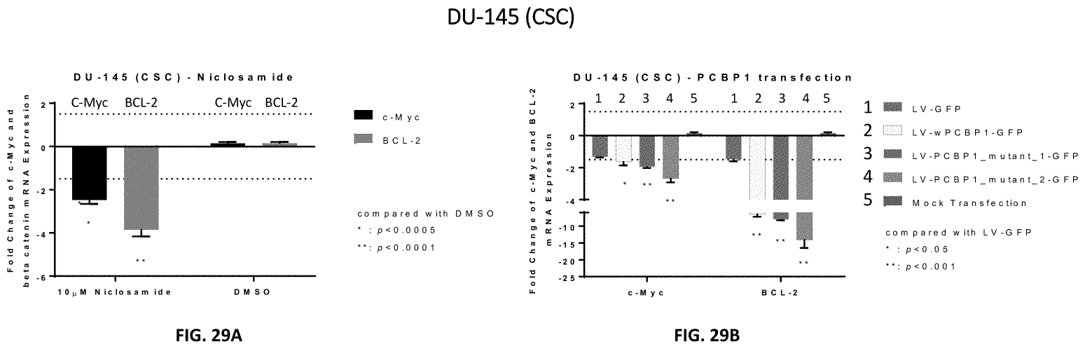

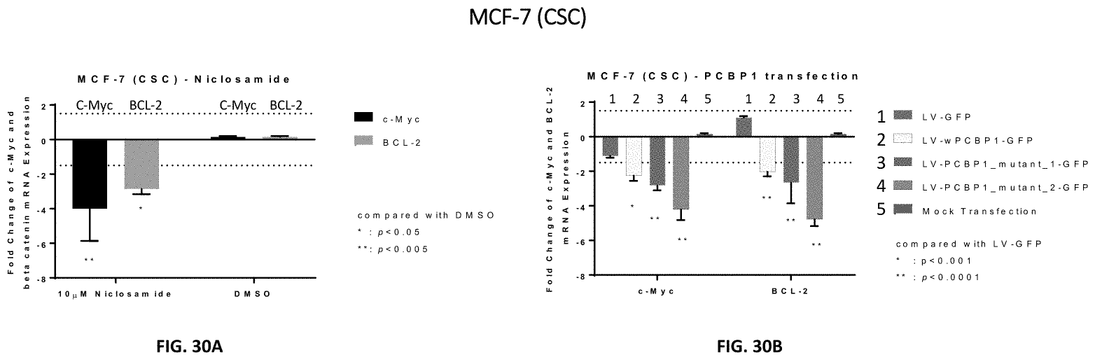

U87-MG (CSC) were used to validate the NF-kB pathway. Cells were lysed using RIPA lysis buffer and 35 mcg cell lysate protein (determined by BCA assay) was loaded per well. Murine anti-c-Myc monoclonal antibody (2ug/mL) and rabbit anti-beta actin antibody (0.5 ug/mL) were used as the primary antibodies. Goat-anti-mouse HRP-conjugated antibody (1:1000) and goat-anti-rabbit HRP-conjugated antibody (1:1000) were used as the secondary antibodies. The expression of c-Myc in the cells as measured by Western Blot was normalized. FIG. 27A. U87-MG (CSC) cells were treated with either DMSA as a control or 10 μM niclosamide. The total RNA was extracted from the cells 48 hours post-transfection, followed by conversion of the RNA to cDNA via RT-PCR. RNA levels were measured by qPRC using gene-specific Taqman probes for c-Myc and BCL-2. GAPDH served as an internal control. FIG. 28A-B shows the c-Myc and BCL-2 expression in U87-MG (CSC) cells treated with either niclosamide or PCBP1 transfection. The expression of c-Myc and BCL-2 in DU-145 (CSC) and MCF-7 (CSC) cells similarly treated is shown in FIG. 29A-B and FIG. 30A-B.

Summary: Both BCL-2 and c-Myc were significantly down-regulated by treatment with PCBP1 (and mutants) or niclosamide. Since niclosamide has been used as an inhibitor of cancer stem cells, these data support comparable effects of PCBP1 gene therapy. These data thus indicate that PCBP1 also acts as a cancer stem cell inhibitor because it can block the expression of CD44 and CD133, possibly via the down-regulation of both the BCL-2 and c-Myc expression, similar to the effects of niclosamide on the NF-kB pathway. These data support use of PCBP1 and mutants in inhibiting and preventing the tumorigenesis during the generation of iPSCs.

### EXAMPLES OF NON-LIMITING EMBODIMENTS OF THE DISCLOSURE

Embodiments, of the present subject matter disclosed herein may be beneficial alone or in combination, with one or more other embodiments. Without limiting the foregoing description, certain non-limiting embodiments of the disclosure, numbered 1 to 24 are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

Embodiment 1. A method of inducing pluripotent stem cells from differentiated cells comprising introducing a vector comprising the nucleic acid sequences of PCBP1 or a mutant or variant thereof or PCBP1 or a mutant or variant thereof and one or more other transcription factors, wherein induction of the pluripotent stem cells is not accompanied by tumorigenesis.

Embodiment 2. The method of embodiment 1, wherein the method is performed in vitro.

Embodiment 3. The method of embodiment 1, wherein the method is performed ex vivo.

Embodiment 4. The method of embodiment 1, wherein the method is performed in vivo.

Embodiment 5. The method of any one of embodiments 1-4, wherein the differentiated cells are mammalian.

Embodiment 6. The method of embodiment 5, wherein the mammalian cells are human.

Embodiment 7. The method of embodiment 5 or 6, wherein the mammalian cells are organ cells, tissue cells, or blood cells.

Embodiment 8. The method of embodiment 7, wherein the tissue cells are retinal or cardiac cells.

Embodiment 9. The method of any of the preceding embodiments, wherein the vector comprises the nucleic acid sequences of PCBP1 or a mutant or variant thereof and at least one of the transcription factors selected from the group consisting of SOX2, OCT4, and KTL4.

Embodiment 10. The method of any of the preceding embodiments, wherein the vector comprises the nucleic acid sequences of PCBP1 or a mutant or variant thereof and at least two of the transcription factors selected from the group consisting of SOX2, OCT4, and KTL4.

Embodiment 11. The method of any of the preceding embodiments, wherein the vector comprises the nucleic acid sequences of PCBP1 or a mutant or variant thereof, SOX2, OCT4, and KTL4.

Embodiment 12. A method of inducing pluripotent stem cells from differentiated cells comprising introducing a vector comprising PCBP1 or a mutant or variant thereof, wherein induction of the pluripotent stem cells is not accompanied by tumorigenesis.

Embodiment 13. The method of embodiment 12, wherein the method is performed in vitro.

Embodiment 14. The method of embodiment 12, wherein the method is performed ex vivo.

Embodiment 15. The method of embodiment 12, wherein the method is performed in vivo.

Embodiment 16. The method of any one of embodiments 12-15, wherein the differentiated cells are mammalian.

Embodiment 17. The method of embodiment 16, wherein the mammalian cells are human.

Embodiment 18. The method of embodiment 16 or 17, wherein the mammalian cells are organ cells, tissue cells, or blood cells.

Embodiment 19. The method of embodiment 18 wherein the tissue cells are retinal or cardiac cells.

Embodiment 20. The method of any one of embodiments 12-19, wherein the vector comprises the nucleic acid sequences of PCBP1 or a mutant or variant thereof and one or more other transcription factors.

Embodiment 21. The method of any one of embodiments 12-20, wherein the vector comprises the nucleic acid sequences of PCBP1 or a mutant or variant thereof and at least one of the transcription factors selected from the group consisting of SOX2, OCT4, and KTL4.

Embodiment 22. The method of any one of embodiments 12-21, wherein the vector comprises the nucleic acid sequences of PCBP1 and at least two of the transcription factors selected from the group consisting of SOX2, OCT4, and KTL4.

Embodiment 23. The method of any one of embodiments 12-22, wherein the vector comprises the nucleic acid sequences of PCBP1, SOX2, OCT4, and KTL4.

Embodiment 24. The method of any one of embodiments 12-23, wherein the vector does not comprise an additional transcription factor.

### INCORPORATION BY REFERENCE

All publications, patents, and patent publications cited are incorporated by reference herein in their entirety for all purposes. The contents of PCT/US2019/016688 file Feb. 5, 2019 are incorporated by reference in their entireties for all purposes.

### REFERENCES

1. J. Krause William (2005-07-01). Krause's Essential Human Histology for Medical Students. Boca Raton: Universal Publishers. ISBN 9781581124682.

2. Optigene, Glossary. http://www.optigen.com/opt9_glossary.html

3. Sohocki, M. M.; Daiger, S. P.; Bowne, S. J.; Rodriquez, J. A.; Northrup, H; Heckenlively, J. R.; Birch, D. G.; Mintz-Hittner, H; Ruiz, R. S.; Lewis, R. A.; Saperstein, D. A.; Sullivan, L. S. (2001). “Prevalence of Mutations Causing Retinitis Pigmentosa and Other Inherited Retinopathies”. Human Mutation. 17 (1): 42-51. doi:10.1002/1098-1004(2001)17:1<42::AID-HUMU5>3.0.CO;2-K. PMC 2585107Freely accessible. PMID 11139241.

4. Elena Ezhkova and Elaine Fuchs. (2010). An eye to treating blindness. Nature. 2010 Jul. 29; 466(7306): 567-568. doi: 10.1038/466567a

5. Schmidt, R., & Plath, K. (2012). The roles of the reprogramming factors Oct4, Sox2 and Klf4 in resetting the somatic cell epigenome during induced pluripotent stem cell generation. Genome Biology, 13(10), 251. http://doi.org/10.1186/gb-2012-13-10-251

6. Yori, J. L., Seachrist, D. D., Johnson, E., Lozada, K. L., Abdul-Karim, F. W., Chodosh, L. A., Ken, R. A. (2011). Krüppel-like Factor 4 Inhibits Tumorigenic Progression and Metastasis in a Mouse Model of Breast Cancer. Neoplasia (New York, N.Y.), 13(7), 601-610.

7. Yu, F., Li, J., Chen, H., Fu, J., Ray, S., Huang, S., . . . Ai, W. (2011). Kruppel-like factor 4 (KLF4) is required for maintenance of breast cancer stem cells and for cell migration and invasion. Oncogene, 30(18), 2161-2172. http://doi.org/10.1038/onc.2010.591

8. Ghanem, L. R., Kromer, A., Silverman, I. M., Chatterji, P., Traxler, E., Penzo-Mendez, A., Liebhaber, S. A. (2016). The Poly(C) Binding Protein Pcbp2 and Its Retrotransposed Derivative Pcbp1 Are Independently Essential to Mouse Development. Molecular and Cellular Biology, 36(2), 304-319. http://doi.org/10.1128/MCB.00936-15

9. M. J. Evans & M. H Kaufman. Establishment in culture of pluripotential cells from mouse embryos. Nature. Vol. 292. 9 Jul. 1981.

10. Wilbur, Beth, editor. The World of the Cell, Becker, W. M., et al., 7th ed. San Francisco, Calif.; 2009.

11. Jomary C et al. Induction of functional photoreceptor phemotype by exogenous CRX expression in mouse tentinal stem cells. Invest Ophthalmol Vis Sci. 2008 Jan; 49 (1): 429-37. Doi:10.1167/iovs. 07-0812

12. Basak et al., The Metastasis-Associated gene PRL-3 is a p53 Target involved in cell-cycle regulation. Mol Cell. 2008 May 9. 30(3):303-314. Doi: 10.1016/j.molcel.2008.04.002

13. Park, S. et al. Limbal Approach-Subretinal Injection of Viral Vector for Gene Therapy in Mice Retinal Pigment Epithelium

14. Lai, Z., Han, I., Zirzow, G., Brady, R O and Reiser, J. Intercellular protein delivery by lentivirus vectors using a herpes simplex virus vp22 fusion protein Proc. Natl. Acad. Sci. USA. 2000; 97: 11297-11302.

15. Chaudhury A, Chander P, Howe P H (2010) Heterogeneous nuclear ribonucleoproteins (hnRNPs) in cellular processes: Focus on hnRNP E1's multifunctional regulatory roles. RNA 16(8): 1449-1462.

16. Friedman D S, O'Colmain B J, Muñoz B, et al; Eye Diseases Prevalence Research Group. Prevalence of age-related macular degeneration in the United States. Arch Ophthalmol. 2004;122(4):564-572.

17. Vingerling J R, Dielemans I, Hofman A, et al. The prevalence of age-related maculopathy in the Rotterdam Study. Ophthalmology. 1995; 102(2): 205-210.

18. Klein R, Knudtson M D, Lee K E, et al. Age-period-cohort effect on the incidence of age-related macular degeneration: the Beaver Dam Eye Study. Ophthalmology. 2008;115(9):1460-1467.

19. Klein R, Klein B E, Lee K E, et al. Changes in visual acuity in a population over a 15-year period: the Beaver Dam Eye Study. Am J Ophthalmol. 2006;142(4):539-549.

20. Lai, Z., et al. “Aquaporin gene therapy corrects Sjögren's syndrome phenotype in mice”. Proceedings of the National Academy of Sciences, May 17, 2016. vol. 113 no. 20.5694-5699, doi: 10.1073/pnas.1601992113.

21. Lu B, Malcuit C, Wang S, et al. Long-term safety and function of RPE from human embryonic stem cells in preclinical models of macular degeneration. Stem Cells. 2009;27:2126-2135.

22. Schwartz et al. (2016) Subretinal Transplantation of Embryonic Stem Cell-Derived Retinal Pigment Epithelium for the Treatment of Macular Degeneration: An Assessment at 4 Years. Invest Ophthalmol Vis Sci. Apr 1;57(5):ORSFc1-9. doi: 10.1167/iovs.15-18681

23. Martin M J, Muotri A, Gage F, Varki A. Human embryonic stem cells express an immunogenic nonhuman sialic acid. Nat Med. 2005;11(2): 228-232.

24. Sakamoto N, Tsuji K, Muul L M, et al. Bovine apolipoprotein B-100 is a dominant immunogen in therapeutic cell populations cultured in fetal calf serum in mice and humans. Blood. 2007;110(2): 501-508.

25. Tezel T H, Kaplan H J, Del Priore L V. Fate of human retinal pigment epithelial cells seeded onto layers of human Bruch's membrane. Invest Ophthalmol Vis Sci. 1999;40:467-476.

26. Del Priore L V, Tezel T H. Reattachment rate of human retinal pigment epithelium to layers of human Bruch's membrane. Arch Ophthalmo1.1998;116:335-341.

27. Gullapalli V K, Sugino I K, Van Patten Y, et al. Retinal pigment epithelium resurfacing of aged submacular human Bruch's membrane. Trans Am Ophthalmol Soc. 2004;102:123-137. discussion 137-138.

28. Sun K, Cai H, Tezel T H, et al. Bruch's membrane aging decreases phagocytosis of outer segments by retinal pigment epithelium. Mol Vis. 2007;13:2310-2319.

29. Afshari F T, Kwok J C, Andrews M R, et al. Integrin activation or alpha 9 expression allows retinal pigmented epithelial cell adhesion on Bruch's membrane in wet age-related macular degeneration. Brain. 2010;133: 448-464.

30. Afshari F T, Fawcett J W. Improving RPE adhesion to Bruch's membrane. Eye. 2009;23(10):1890-1893.

31. Fang I M, Yang C H, Yang C M, Chen M S. Overexpression of integrin alpha6 and beta4 enhances adhesion and proliferation of human retinal pigment epithelial cells on layers of porcine Bruch's membrane. Exp Eye Res. 2009;88:12-21.

32. Del Priore L V, Ishida O, Johnson E W, et al. Triple immune suppression increases short-term survival of porcine fetal retinal pigment epithelium xenografts. Invest Ophthalmol Vis Sci. 2003;44(9):4044-4053.

33. Lai C C, Gouras P, Dio K, et al. Local immunosuppression prolongs survival of RPE xenografts labeled by retroviral gene transfer. Invest Ophthalmol Vis Sci. 2000;41(10):3134-3141.

34. He S, Wang H M, Ogden T E, Ryan S J. Transplantation of cultured human retinal pigment epithelium into rabbit subretina. Graefes Arch Clin Exp Ophthalmol. 1993;231(12):737-742.

35. Lai, Z., eta. “Aquaporin gene therapy corrects Sjögren's syndrome phenotype in mice”. Proceedings of the National Academy of Sciences, May 17,2016. vol. 113 no. 20.5694-5699.

36. Lai, Z, and Brady, R O. Gene transfer into the central nervous system in vivo using a recombinant lentivirus vector. Journal of Neuroscience Research. 2002; 67: 363-371 (correspondent author).

37. Lai Z., 2002: Design of an HIV-1 Lentiviral-Based Gene-Trap Vector to Detect Developmentally Regulated Genes in Mammalian Cells. Proceedings of the National Academy of Sciences of the United States of America Vol. 99, No. 6 (Mar. 19, 2002), pp. 3651-3656.

38. Yin H, Cabrera J, Lai Z, et al., Association of bone morphogenetic protein 6 with exocrine gland dysfunction in patients with Sjögren's syndrome and in mice. Arthritis Rheum. 2013 Dec; 65 (12):3228-38.

39. Lai Z., et al., PNAS, 2002: Design of an HIV-1 Lentiviral-Based Gene-Trap Vector to Detect Developmentally Regulated Genes in Mammalian Cells. Proceedings of the National Academy of Sciences of the United States of America Vol. 99, No. 6 (Mar. 19, 2002), pp. 3651-3656.

40. Chen J X, Krane M, Deutsch M A, et al., Inefficient reprogramming of fibroblasts into cardiomyocytes using Gata4, Mef2c, and Tbx5. Circ Res. 2012 Jun 22;111(1):50-5.

41. Ma H, Wang L, Liu J, and Qian L. Direct Cardiac Reprogramming as a Novel Therapeutic Strategy for Treatment of Myocardial Infarction Methods Mol Biol. 2017; 1521: 69-88.

42. Heart Disease and Stroke Statistics—2017 Update

43. Takahashi K, Tanabe K, Ohnuki M, Narita M, Ichisaka T, Tomoda K, Yamanaka S. Induction of pluripotent stem cells from adult human fibroblasts by defined factors. Cell. 2007 Nov 30;131(5):861-72.

44. Zhao Y, Londono P, Cao Y, et al., High-efficiency reprogramming of fibroblasts into cardiomyocytes requires suppression of profibrotic signalling. Nat Commun. 2015 Sep 10;6:8243

45. Wang H. et al. PCBP1 suppresses the translation of metastasis-associated PRL-3 phosphatase. Cancer Cell. 2010 Jul 13;18(1):52-62. doi: 10.1016/j.ccr.2010.04.028.

46. Lai, Z., Han, I., Zirzow, G., Brady, R. O. & Reiser, J. Intercellular delivery of a herpes simplex virus VP22 fusion protein from cells infected with lentiviral vectors. Proc. Natl. Acad. Sci. U. S. A. 97, 11297-302 (2000).

47. Beltrami A P, et al. Adult cardiac stem cells are multipotent and support myocardial regeneration. Cell. 2003;114(6):763-76. Epub 2003/09/25. pmid:1450557514.

48. Bearzi, C. et al. Human cardiac stem cells. Proceedings of the National Academy of Sciences. 14068-14073, doi: 10.1073/pnas.0706760104

49. Barile, L. et al. Cardiac stem cells: isolation, expansion and experimental use for myocardial regeneration. Nature Clinical Practice Cardiovascular Medicine (2007) 4, S9-S14

50. Barile, L. et al. Human Cardiospheres as a Source of Multipotent Stem and Progenitor Cells. Stem Cells International. Volume 2013 (2013), Article ID 916837.

51. Malliaras, K. Intracoronary Cardiosphere-Derived Cells After Myocardial Infarction-Evidence of Therapeutic Regeneration in the Final 1-Year Results of the CADUCEUS Trial (CArdiosphere-Derived aUtologous stem CElls to reverse ventricUlar dySfunction). Journal of the American College of Cardiology. Volume 63, Issue 2, January 2014. DOI: 10.1016/j.jacc.2013.08.724

52. Moore-Morris, T. et al. Resident fibroblast lineages mediate pressure overload-induced cardiac fibrosis. The Journal of Clinical Investigation. 2014;124(7):2921-2934. doi:10.1172/JCI74783.

53. Masino A et al. Transcriptional Regulation of Cardiac Progenitor Cell Populations. Circulation Research. August 20, 2004. DOI: 10.1161/01.RES.0000138302.02691.be

54. Reiser, J., Lai, Z., Zhang, X. Y. & Brady, R. O. Development of multigene and regulated lentivirus vectors. J. Virol. 74, 10589-99 (2000).

55. Lai, Z. & Brady, R. O. Gene transfer into the central nervous system in vivo using a recombinanat lentivirus vector. J. Neurosci. Res. 67, 363-371 (2002).

56. Lai, Z., Han, I., Park, M. & Brady, R. O. Design of an HIV-1 lentiviral-based gene-trap vector to detect developmentally regulated genes in mammalian cells. Proc. Natl. Acad. Sci. U. S. A. 99, 3651-6 (2002).

57. Kolk, M V. et al. LAD-ligation: a murine model of myocardial infarction. J Vis Exp. 2009 Oct 14;(32). pii: 1438. doi: 10.3791/1438.

58. Jaski B E et al., Calcium upregulation by percutaneous administration of gene therapy in cardiac disease (CUPID Trial), a first-inhuman phase 1/2 clinical trial. J Card Fail. 2009 Apr;15(3):171-81.

59. Merkle, F T, et al. (2017) Human pluripotent stem cells recurrently acquire and expand dominant negative P53 mutations. Nature 545: 229-233.

60. Di Stefano B. et al. (2016). C/EBPα creates elite cells for iPSC reprogramming by upregulating Klf4 and increasing the levels of Lsd1 and Brd4. Apr;18(4):371-81. doi: 10.1038/ncb3326. Epub 2016 Mar 14.

61. Collin, J. et al. (2019) CRX Expression in Pluripotent Stem Cell-Derived Photoreceptors Marks a Transplantable Subpopulation of Early Cones. Stern Cells. 2019 May;37(5):609-622. doi: 10.1002/stem.2974. Epub 2019 Jan 30.

62. Samuel et al 2019, Appropriately differentiated ARPE-19 cells regain phenotype and gene expression profiles similar to those of native RPE cells. Mol. Vis. 23:60-89.

63. Hazim et al 2019, Rapid differentiation of the human RPE cell line, ARPE-19, induced by nicotinamide. Exp Eye Res, 179:18-24.

64. Warburton et al 2004, Proteomic Comparison of Dedifferentiated and Differentiated ARPE-19 Cells with Human Retinal Pigment Epithelial Cells. Invest Ophthal Vis Sci 45:634.

65. Ahmado et al 2011. Induction of Differentiation by Pyruvate and DMEM in the Human Retinal Pigment Epithelium Cell Line ARPE-19. Invest Ophthal Vis Sci 52:7148-7159.

66. Bouchlaka, Myriam N et al. (2010) Immunotherapy following hematopoietic stem cell transplantation: potential for synergistic effects.” Immunotherapy vol. 2,3: 399-418. doi:10.2217/imt.10.20 (https://www.ncbi.nlm.nih.gov/pmc/articles/PMC3492055/).

67. Bricker-Anthony C & Rex T S (2015). Neurodegeneration and Vision Loss after Mild Blunt Trauma in the C57B1/6 and DBA/2J Mouse” PLoS ONE 10(7): e0131921. doi:10.1371/journal. pone.0131921.

68. Hines-Beard, Jessica et al. (2012) A mouse model of ocular blast injury that induces closed globe anterior and posterior pole damage.” Experimental eye research vol. 99: 63-70. doi:10.1016/j.exer.2012.03.013

69. Chowers, G. et al. (2017) Course of Sodium Iodate-Induced Retinal Degeneration in Albino and Pigmented Mice, Retina, IOVS:April Vol. 58.No. 4; 2239 -2249.

70. Pennesi et al. (2012) Animal models of age related macular degeneration, Mol Aspects Med. August; 33(4): 487-509. doi:10.1016/j.mam.2012.06.003.

71. Turgut et al. (2017). Experimental Animal Models for Retinal and Choroidal Diseases; Adv Ophthalmol Vis Syst., 7(4): 00232

72. Wang L, Liu Z, Yin C, et al. (2015) Stoichiometry of Gata4, Mef2c, and Tbx5 influences the efficiency and quality of induced cardiac myocyte reprogramming. Circ Res. 2015;116(2):237-244. doi:10.1161/CIRCRESAHA.116.305547.

73. Liu et al. (2013) Oncogene Jan 31;32(5):544-53. doi: 10.1038/onc.2012.85. Epub 2012 Apr 2.

74. Pan, Jing-Xuan et al. “Niclosamide, an old antihelminthic agent, demonstrates antitumor activity by blocking multiple signaling pathways of cancer stem cells.” Chinese journal of cancer vol. 31,4 (2012): 178-84. doi:10.5732/cjc.011.1029

1. A method of inducing pluripotent stem cells from differentiated cells comprising introducing a vector comprising the nucleic acid sequences of PCBP1 or a mutant or variant thereof or PCBP1 or a mutant or variant thereof and one or more other transcription factors, wherein induction of the pluripotent stem cells is not accompanied by tumorigenesis.

2. The method of claim 1, wherein the method is performed in vitro.

3. The method of claim 1, wherein the method is performed ex vivo.

4. The method of claim 1, wherein the method is performed in vivo.

5. The method of claim 1, wherein the differentiated cells are mammalian.

6. The method of claim 5, wherein the mammalian cells are human.

7. The method of claim 5, wherein the mammalian cells are organ cells, tissue cells, or blood cells.

8. The method of claim 7, wherein the tissue cells are retinal or cardiac cells.

9. The method of claim 1, wherein the vector comprises the nucleic acid sequences of PCBP1 or a mutant or variant thereof and at least one of the transcription factors selected from the group consisting of SOX2, OCT4, and KTL4.

10. The method of claim 1, wherein the vector comprises the nucleic acid sequences of PCBP1 or a mutant or variant thereof and at least two of the transcription factors selected from the group consisting of SOX2, OCT4, and KTL4.

11. The method of claim 1, wherein the vector comprises the nucleic acid sequences of PCBP1 or a mutant or variant thereof, SOX2, OCT4, and KTL4.

12. A method of inducing pluripotent stem cells from differentiated cells comprising introducing a vector comprising PCBP1 or a mutant or variant thereof, wherein induction of the pluripotent stem cells is not accompanied by tumorigenesis.

13. The method of claim 12, wherein the method is performed in vitro.

14. The method of claim 12, wherein the method is performed ex vivo.

15. The method of claim 12, wherein the method is performed in vivo.

16. The method of claim 12, wherein the differentiated cells are mammalian.

17. The method of claim 16, wherein the mammalian cells are human.

18. The method of claim 16, wherein the mammalian cells are organ cells, tissue cells, or blood cells.

19. The method of claim 18 wherein the tissue cells are retinal or cardiac cells.

20. The method of claim 12, wherein the vector comprises the nucleic acid sequences of PCBP1 or a mutant or variant thereof and one or more other transcription factors.

21. The method of claim 20, wherein the vector comprises the nucleic acid sequences of PCBP1 or a mutant or variant thereof and at least one of the transcription factors selected from the group consisting of SOX2, OCT4, and KTL4.

22. The method of claim 20, wherein the vector comprises the nucleic acid sequences of PCBP1 and at least two of the transcription factors selected from the group consisting of SOX2, OCT4, and KTL4.

23. The method of claim 20, wherein the vector comprises the nucleic acid sequences of PCBP1, SOX2, OCT4, and KTL4.

24. The method of claim 20, wherein the vector does not comprise an additional transcription factor.


SEQUENCE LISTING (5 sequences)

SEQ ID NO: 1 - DNA, 1200 bp, Homo sapiens
gttcgttgcaacaaattgatgagcaatgcttttttataatgccaactttgtacaaaaaagttggcatggatgccggtgtgactgaaagtggactaaatgtgactctcaccattcggcttcttatgcacggaaaggaagtaggaagcatcattgggaagaaaggggagtcggttaagaggatccgcgaggagagtggcgcgcggatcaacatctcggaggggaattgtccggagagaatcatcactctgaccggccccaccaatgccatctttaaggctttcgctatgatcatcgacaagctggaggaagatatcaacagctccatgaccaacagtaccgcggccagcaggcccccggtcaccctgaggctggtggtgccggccacccagtgcggctccctgattgggaaaggcgggtgtaagatcaaagagatccgcgagagtacgggggcgcaggtccaggtggcgggggatatgctgcccaactccaccgagcgggccatcaccatcgctggcgtgccgcagtctgtcaccgagtgtgtcaagcagatttgcctggtcatgctggagacgctctcccagtctccgcaagggagagtcatgaccattccgtaccagcccatgccggccagctccccagtcatctgcgcgggcggccaagatcggtgcagcgacgctgcgggctacccccatgccacccatgacctggagggaccacctctagatgcctactcgattcaaggacaacacaccatttctccgctcgatctggccaagctgaaccaggtggcaagacaacagtctcactttgccatgatgcacggcgggaccggattcgccggaattgactccagctctccagaggtgaaaggctattgggcaagtttggatgcatctactcaaaccacccatgaactcaccattccaaataacttaattggctgcataatcgggcgccaaggcgccaacattaatgagatccgccagatgtccggggcccagatcaaaattgccaacccagtggaaggctcctctggtaggcaggttactatcactggctctgctgccagtattagtctggcccagtatctaatcaatgccaggctttcctctgagaagggcatggggtgcagctacccaactttcttgtacaaagttggcattataagaaagcattgcttatcaatttgttgcaacgaac

SEQ ID NO: 2 - protein, 356 aa, Homo Sapiens
MDAGVTESGLNVTLTIRLLMHGKEVGSIIGKKGESVKRIREESGARINISEGNCPERIITLTGPTNAIFKAFAMIIDKLEEDINSSMTNSTAASRPPVTLRLVVPATQCGSLIGKGGCKIKEIRESTGAQVQVAGDMLPNSTERAITIAGVPQSVTECVKQICLVMLETLSQSPQGRVMTIPYQPMPASSPVICAGGQDRCSDAAGYPHATHDLEGPPLDAYSIQGQHTISPLDLAKLNQVARQQSHFAMMHGGTGFAGIDSSSPEVKGYWASLDASTQTTHELTIPNNLIGCIIGRQGANINEIRQMSGAQIKIANPVEGSSGRQVTITGSAASISLAQYLINARLSSEKGMGCS

SEQ ID NO: 3 - DNA, 602 bp, Homo sapiens
attctcatcgtaaatcaggctcacttccattggctgcatacggtggagtgatgtgaccatatgtcacttgagcattacacaaatcctaatgagctaaaaatatgtttgttttagctaattgacctctttggccttcataaagcagttggtaaacatcctcagataatgatttccaaagagcagattgtgggtctcacctgtgcagagaaagcccacgtccctgagaccaccttctccaggcctactgaggcacattctcatcgtaaatcaggctcacttccattggctgcatacggtggagtgatgtgaccatatgtcacttgagcattacacaaatcctaatgagctaaaaatatgtttgttttagctaattgacctctttggccttcataaagcagttggtaaacatcctcagataatgatttccaaagagcagattgtgggtctcacctgtgcagagaaagcccacgtccctgagaccaccttctccaggcctactgaggcacacaggggcgcctgcctgctgcccgctcagccaaggcggtgttgctggaacaggggcgcctgcctgctgcccgctcagccaaggcggtgttgctgga

SEQ ID NO: 4 - DNA, 424 bp, Artificial
  Synthesized
ggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattg

SEQ ID NO: 5 - DNA, 424 bp, Artificial; identical to SEQ ID NO: 4
  Synthesized